(19) United States
(12) Patent Application Publication
(10) Pub. No.: US 2013/0211076 A1
(43) Pub. Date: Aug. 15, 2013

(54) Method for preparing substituted N-(3-amino-quinoxalin-2-yl)-sulfonamides and their intermediates N-(3-chloro-quinoxalin-2-yl)-sulfonamides

(71) Applicants: Danig Pohin, Lausanne (CH); Dominique Swinnen, Beaumont (FR)

(72) Inventors: Danig Pohin, Lausanne (CH); Dominique Swinnen, Beaumont (FR)

(73) Assignee: MERCK SERONO S.A. GENEVA, GENEVA (CH)

(21) Appl. No.: 13/880,445

(22) PCT Filed: Oct. 18, 2011
(86) PCT No.: PCT/EP2011/068152

§ 371 Date: Apr. 19, 2013

Related U.S. Application Data
(60) Provisional application No. 61/424,933, filed on Dec. 20, 2010.

(30) Foreign Application Priority Data
Oct. 20, 2010 (EP) 10188170.4

(51) Int. Cl. C07D 241/44; C07D 409/12; C07D 241/22; C07D 405/12; C07D 417/12; C07D 401/12; C07D 403/12; C07D 405/14

(52) U.S. Cl. CPC C07D 241/44; C07D 403/12; C07D 409/12; C07D 405/14; C07D 405/12; C07D 417/12; C07D 401/12; C07D 241/22
USPC 544/58.2; 544/356; 544/119; 544/336

(57) ABSTRACT

The present invention provides a new synthesis for preparing N-(3-amino-quinoxalin-2-yl)-sulfonamides of general formulae (I) or (I′) and intermediates sulfonamides of formula (II) or (II′):

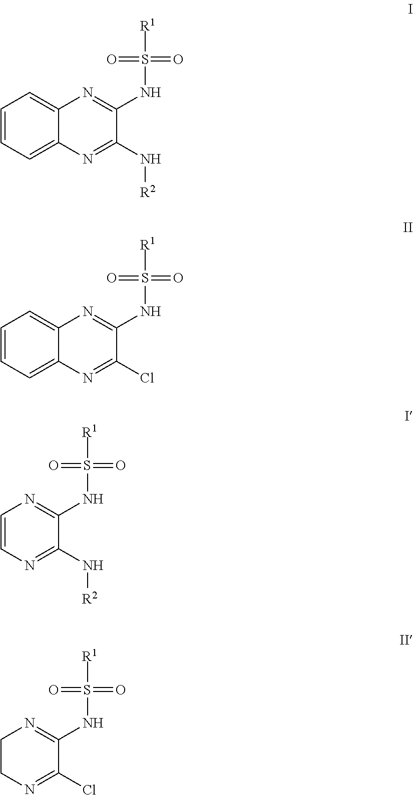

### SUMMARY OF THE INVENTION

The present invention provides a new synthesis for preparing N-(3-amino-quinoxalin-2-yl)-sulfonamides of general formula (I) and its intermediate N-(3-chloro-quinoxalin-2-yl)-sulfonamides of formula (II). The compounds of formulae (I) and (II) are useful building blocks, in particular in the synthesis of drugs.

### FIELD OF THE INVENTION

The present invention is related to a new synthesis for preparing N-(3-amino-quinoxalin-2-yl)-sulfonamides of general formulae (I) and (I′), and their intermediates N-(3-chloro-quinoxalin-2-yl)-sulfonamides of formulae (II) and (II′):

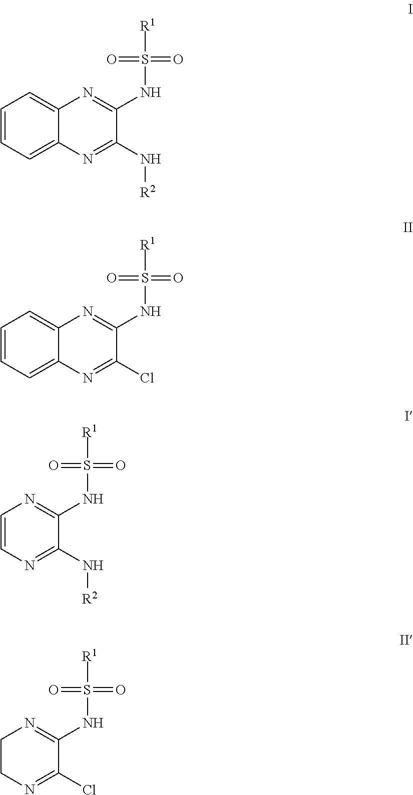

R¹ is selected from the group consisting of A, C₃-C₈-cylcoalkyl, Het, and Ar.
R² is selected from the group consisting of Ar and Het.
Ar denotes a monocyclic or bicyclic, aromatic carbocyclic ring having 6 to 14 carbon atoms, which is unsubstituted or monosubstituted, disubstituted or trisubstituted by Hal, CF₃, OCF₃, NO₂, CN, perfluoroalkyl, A, —OR⁶, —NHR⁶, —COR^{E}, —CONHR⁶, —CON(R⁶)₂, —NR⁶COR⁶, —NR⁶CO₂R⁶, —NR⁶SO₂A, NR⁶CONR′R″, —COOR⁶, —SO₂A, —SO₂NR⁶A, —SO₂Het, —SO₂NR⁶Het, Ar, Het, —NR⁶SO₂NR⁶Het, COHet, COAr, or C₃-C₈-cycloalkyl.
Het denotes a monocyclic or bicyclic saturated, unsaturated or aromatic heterocyclic ring having 1 to 4 N, O and/or S atoms and/or 1 group selected from CO, SO or SO₂, which is unsubstituted or monosubstituted, disubstituted or trisubstituted by Hal, CF₃, OCF₃, NO₂, CN, perfluoroalkyl, A, —OR⁶, —NHR⁶, —COR^{E}, —CONHR⁶, —CON(R⁶)₂, —NR⁶COR⁶, —NR⁶CO₂R⁶, —NR⁶SO₂A, NR⁶CONR′R″, —COOR⁶, —SO₂A, —SO₂NR⁶A, —SO₂Het, —SO₂NR⁶Het, Ar, Het, —NR⁶SO₂NR⁶Het, or C₃-C₈-cycloalkyl.
A is a branched or linear alkyl having 1 to 12 C-atoms, wherein one or more, preferably 1 to 7H-atoms may be replaced by Hal, Ar, Het, OR⁶, CN, NR⁶COA, CONR′R″, COOR⁶ or NR′R″ and wherein one or more, preferably 1 to 7 non-adjacent CH₂-groups may be replaced by O, NR⁶ or S and/or by —CH═CH— or —C≡C— groups, or denotes cycloalkyl, cycloalken or cycloalkylalkylen having 3-7 ring C atoms wherein the cycloalkylen is optionally substituted by 1 to 3 groups selected from OR⁶, Hal, Ar, Het, CN, NR⁶COA, CONR′R″, COOR⁶;
R′, R″ denote independently from each other H, A, Ar, or Het,

### R⁶ is H, A.

The method employs commercially available, or easily obtainable, starting compounds.

### BACKGROUND OF THE INVENTION

The synthetic approaches for preparing N-(3-amino-quinoxalin-2-yl)-sulfonamides (I) are well known. Examples from the prior art reports the reaction of the 2,3-dichloro-quinoxaline (commercially available or easily obtainable from commercially available starting compounds, scheme 1) with the sulfonamide of formula (III) wherein R1 is a aryl or heteroaryl group, to give the intermediate (II) (Scheme 1, Step 1). In a second step, the intermediate N-(3-chloro-quinoxalin-2-yl)-sulfonamides of formula (II) is converted to the N-(3-amino-quinoxalin-2-yl)-sulfonamides (I) by reaction with an amine of formula (IV) wherein R² is a aryl or heteroaryl group (Scheme 1, Step 2).

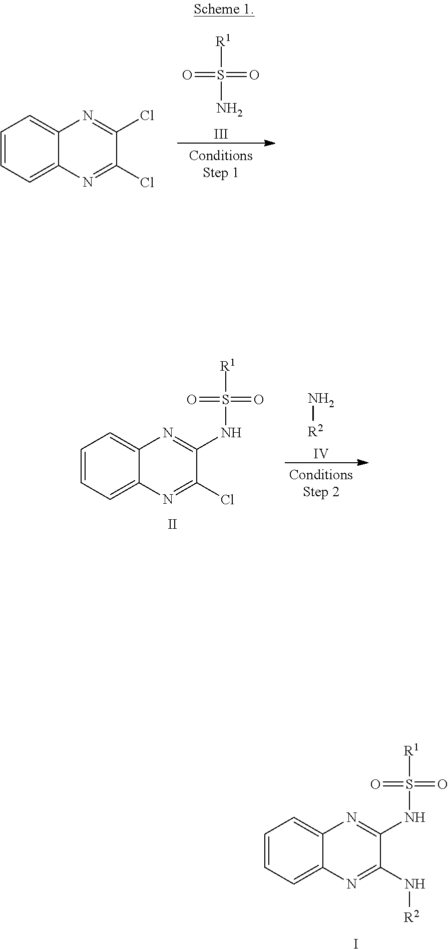

Several documents quote the transformation of the 2,3-dichloro-quinoxaline with sulfonamides of formula (III) into N-(3-chloro-quinoxalin-2-yl)-sulfonamides of formula (II) in the presence of carbonates as bases (e.g. K₂CO₃ or Cs₂CO₃) in polar aprotic solvents such as DMSO, DMF, NMP or DMA (References 1-9), Scheme 2.

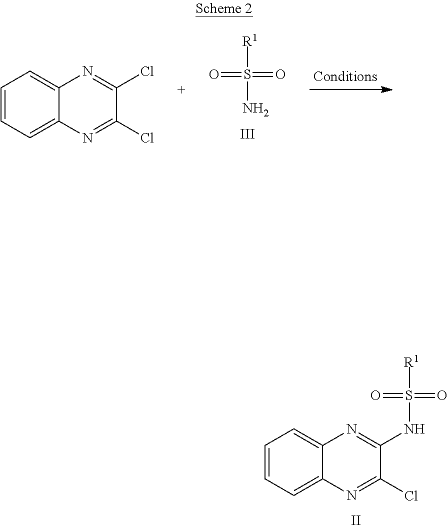

For example, the patent application (WO 2007023186 A1, Reference 2) described the reaction of the 2,3-dichloro-quinoxaline with a compound of Formula (III) wherein R¹ is a phenyl group, i.e. phenylsulfonamide, with potassium carbonate in DMA at 135° C. (80% yield). The same compound was prepared by S. V. Litvinenko et al. (Reference 7) using potassium carbonate in DMF at reflux.

A further example for the synthesis of compounds of Formula (II), wherein R¹ is dichlorophenyl, can be found in WO 2005021513 A1 (Reference 4, example 10 step a, p27) In this example, cesium carbonate is used as a base for the transformation.

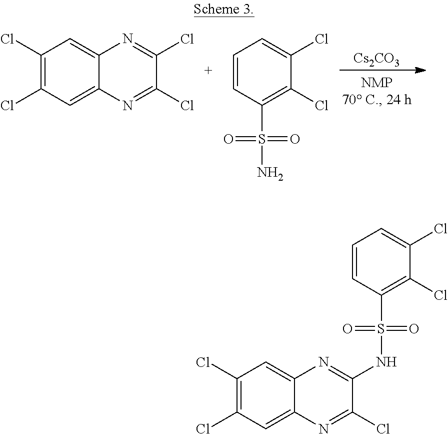

For the formation of N-(3-chloro-quinoxalin-2-yl)-sulfonamides of formula (II), the above methods found in the literature described the use of carbonate bases such as potassium or cesium carbonate. These conditions may need long reaction time or higher temperature for completion. In addition, these reaction conditions may cause the formation of undesired by-products or impurities that are difficult or expensive to remove.

The present invention provides a new method for the synthesis of compounds of Formula (I), wherein step I in scheme 1 does not require the use of the carbonates as a base, but the use of alkali metal hydroxide, particularly lithium hydroxide as a base. Use of an alkali metal hydroxide improves the purity profile and the yield. In addition, use of an alkali metal hydroxide allows to have similar reaction time at lower temperature or to have decreased reaction time.

The second step (scheme 1) consisting in the transformation of compounds of Formula (II) into compounds of Formula (I) is reported in the literature (References 1, 2, 8, 9). These reports often disclosed the reaction at elevated temperature in polar solvents such as DMA, DMF, NMP, DMSO or EtOH, or alternatively, aprotic non polar solvents such as toluene or xylene. Alternative conditions are the use of acetic acid in DMA.

For example, the patent application WO 2007023186 A1, (Reference 2) described the reaction of 4-cyano-N-(3-chloro-quinoxalin-2-yl)-sulfonamides with the 3,5-dimethoxy aniline (IV) in EtOH heated at 100° C. overnight (50% yield), (scheme 4).

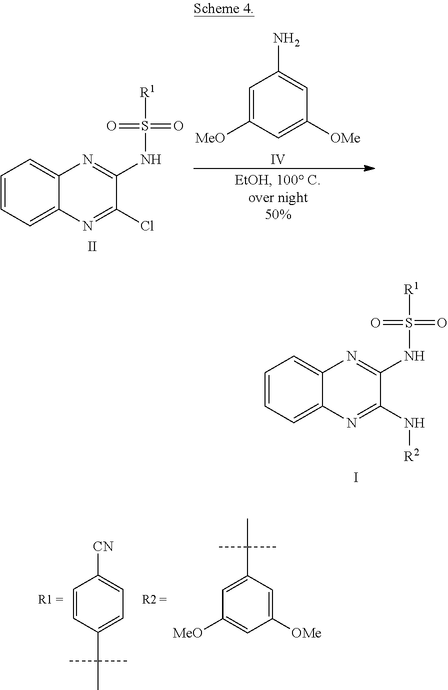

WO 2008127594, (Reference 8, example 373, p434) described the reaction of a compound of Formula (II) wherein R¹ is phenyl with the 4-fluoro aniline in DMA at 120° C., during 25 minutes, under microwave irradiation (62% yield), (Scheme 5).

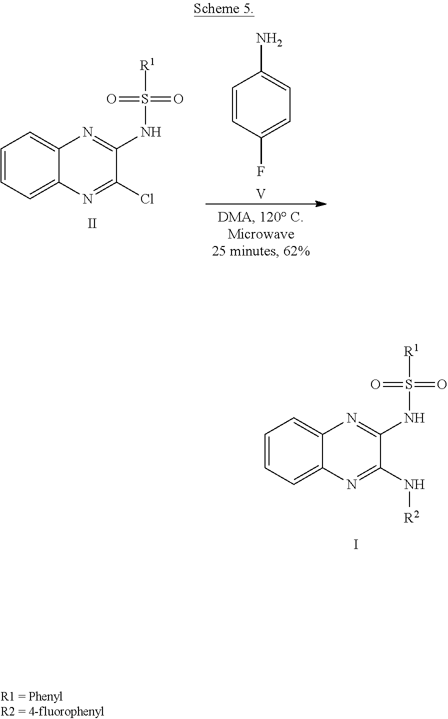

WO 2008127594, (Reference 8, example 14, p379) also described the reaction of N-(3-chloro-quinoxalin-2-yl)-sulfonamides (II) wherein R¹ is a 3 nitro-phenyl with the 3,5-dimethoxy-aniline in xylene at 150° C. (70% yield), (Scheme 6).

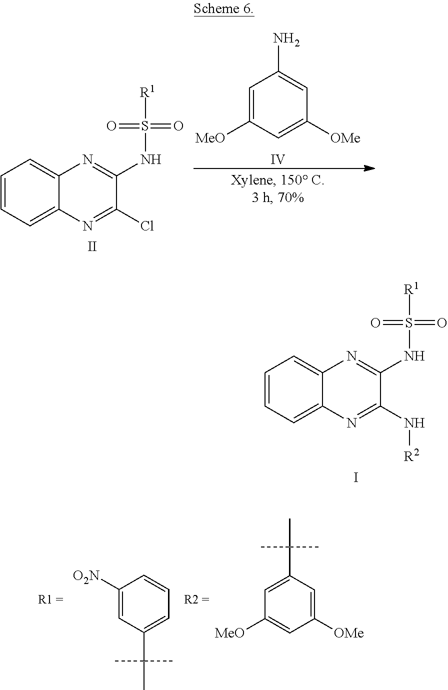

In the formation of N-(3-amino-quinoxalin-2-yl)-sulfonamides of formula (I), the above methods found in the literature involve the heating of an amine of formula NH₂R² in different solvent without bases, or with acetic acid. These conditions may need long reaction time or higher temperature for completion. In addition, these reaction conditions may cause the formation of undesired by-products or impurities that are difficult or expensive to remove.

The present invention provides a new method requiring the use of a pyridine base, preferably the 2,6-dimethylpyridine (lutidine). The use of this base led to improved purity profile and/or improved yields. Also, these conditions allow to have similar reaction time at lower temperature or to have decreased reaction time.

### DESCRIPTION OF THE INVENTION

The present invention provides improved conditions for the preparation of N-(3-amino-quinoxalin-2-yl)-sulfonamides of general formulae (I) and (I′), and their intermediates N-(3-chloro-quinoxalin-2-yl)-sulfonamides of formulae (II) and (II′).

In particular, the present invention provides a new method for the first step (scheme 7) that use of alkali metal hydroxide as a base, improving the purity profile, the yield and allowing to reach excellent yields and conversions at lower temperature compared to the use of other bases such as carbonates, or allowing to reach excellent yields and conversions at the same temperature but in shorter reaction time. The preferred conditions are the ones using lithium hydroxide as the base.

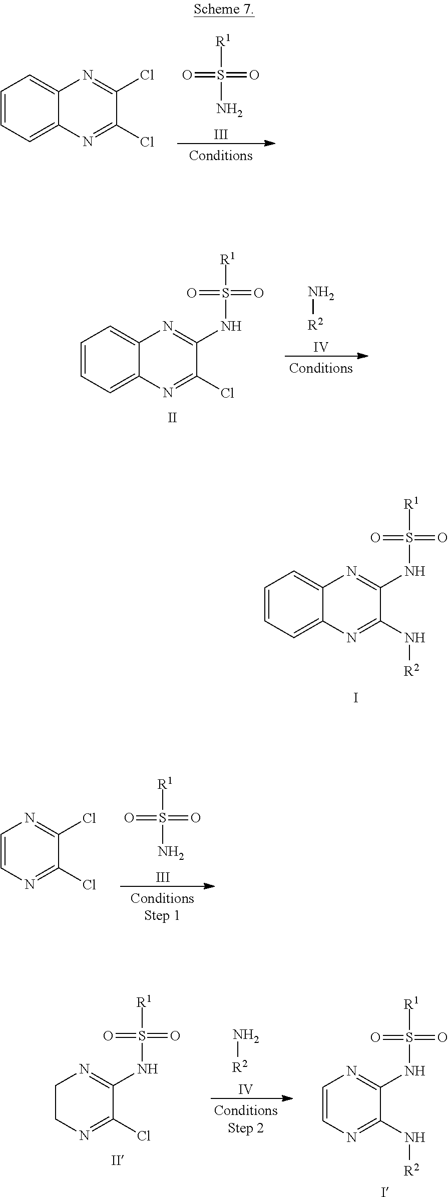

In addition, the present invention provides a new method for the second step (Scheme 6) that use a pyridine base, preferably 2,6-dimethylpyridine (lutidine), improving the purity profile, the yield and allowing to reach excellent yields and conversions at lower temperature compared to the conditions described in the literature, or allowing to reach excellent yields and conversions at the same temperature but in shorter reaction time.

The alkali metal hydroxide bases used in the first step of the synthesis are preferably selected from NaOH, KOH, and LiOH.

An aprotic solvent denotes an organic solvent which does not exchange proton, or “H atom” with the products which are dissolved in it. Aprotic solvents comprises polar aprotic solvents and apolar aprotic solvents.

Examples of polar aprotic solvents are Dichloromethane (DCM), Tetrahydrofuran (THF), Ethyl acetate, Acetone, Dimethylformamide (DMF), Acetonitrile (MeCN), Dimethyl sulfoxide (DMSO), dimethylacetamide (DMA), N-methylpyrrolidone (NMP).

The crude purity of a compound e.g. compounds of Formula (II) or compounds of Formula (I), denotes the ratio of said compounds compared to the other impurities or by-products obtained in the crude mixture, before the purification step. The crude purity is preferably determined using commonly used analytical methods like HPLC (High performance liquid chromatography), GC (Gaz chromatography), GC-MS (Gaz chromatography couple with Mass spectrometry), SFC (supercritical fluid chromatography). These methods may include or not the use of internal references.

Ar preferably denotes a monocyclic or bicyclic, aromatic carbocyclic ring having 6 to 14 carbon atoms, which may be monosubstituted, disubstituted or trisubstituted by:
Hal,
—C₁-C₆-alkyl, optionally substituted by 1 to 3 Hal, OH, OC₁-C₆-alkyl, —(CH₂—CH₂—O)_{q}CH₃, or —(CH₂—CH₂—O—)_{q}H,
OC₁-C₆-alkyl optionally substituted by 1 to 3 Hal, OH, OC₁-C₆-alkyl, —(CH₂—CH₂—O)_{q}CH₃, or —(CH₂—CH₂—O—)_{q}H,
CF₃,
OCF₃,
—NO₂,
—CN,
—(CH₂)ₙNH(C₁-C₆-alkyl),
—(CH₂)ₙCO(C₁-C₆-alkyl),
—(CH2)nCONH(C₁-C₆-alkyl),
—(CH2)nCON(C1-C6-alkyl)2,
—(CH₂)ₙCON(C₁-C₆-alkyl)₂,
—(CH₂)ₙNHCO₂(C₁-C₆-alkyl),
—(CH₂)ₙNHCO(C₁-C₆-alkyl),
—(CH₂)ₙCOHet,
—(CH₂)ₙCOAr,
—(CH₂)ₙN(C₁-C₆-alkyl)(CH₂)ₙAr,
—(CH₂)ₙN(C₁-C₆-alkyl)(CH₂)ₙHet,
n is independently 0, 1, 2 or 3, preferably 0 or 1.
q is independently 0, 1, 2 or 3, preferably 1 or 2.
   More preferably, Ar denotes one of the following groups:

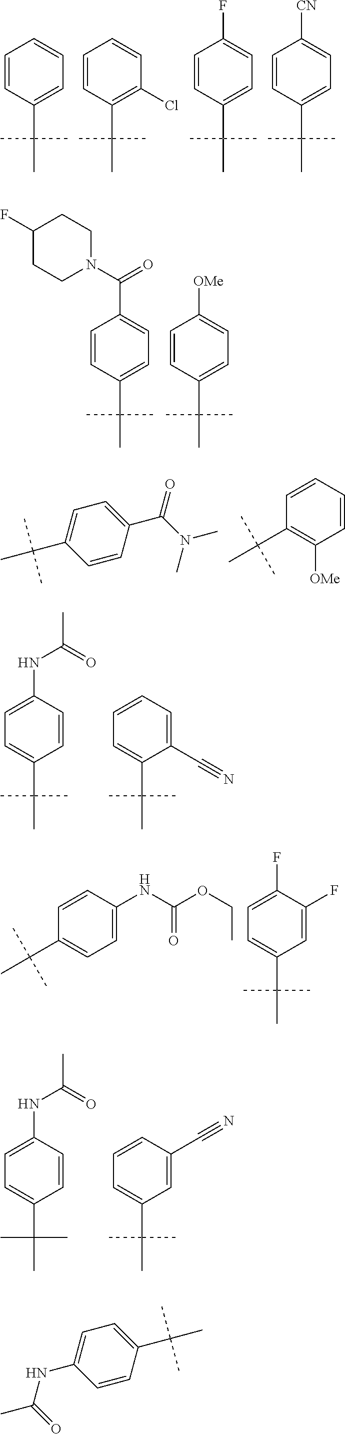

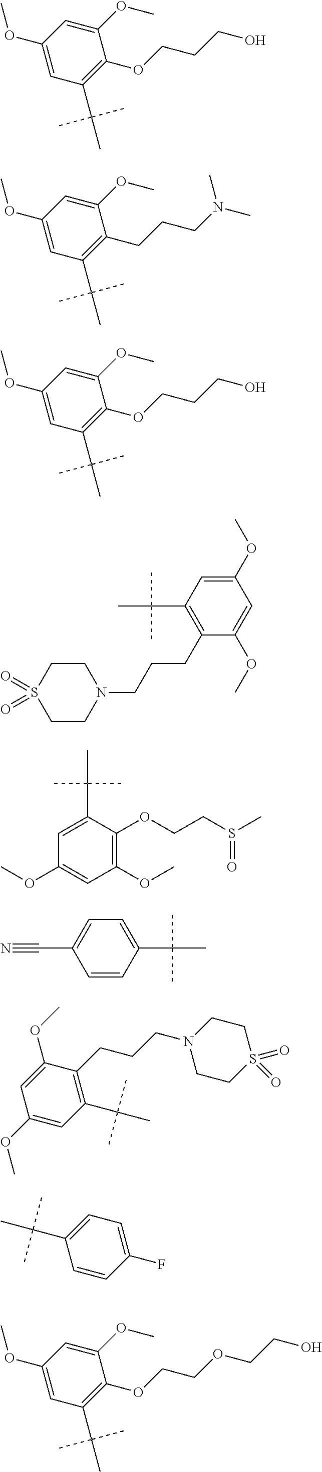

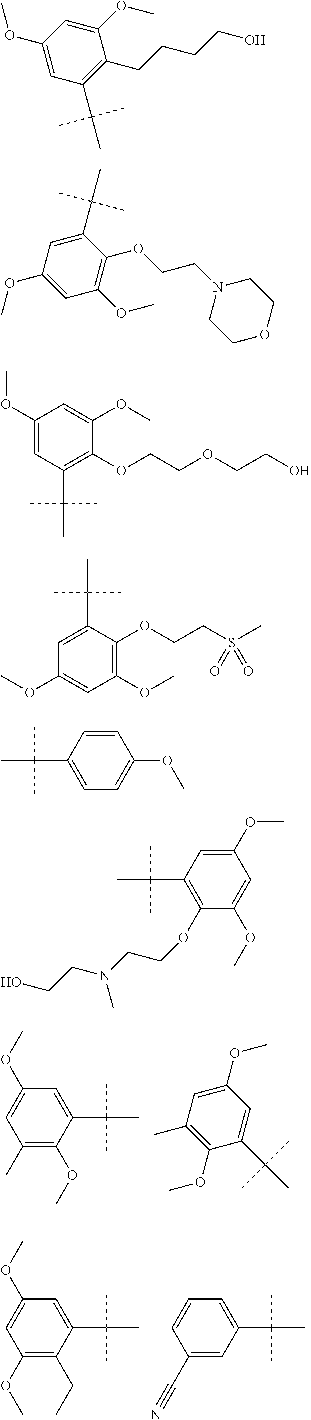

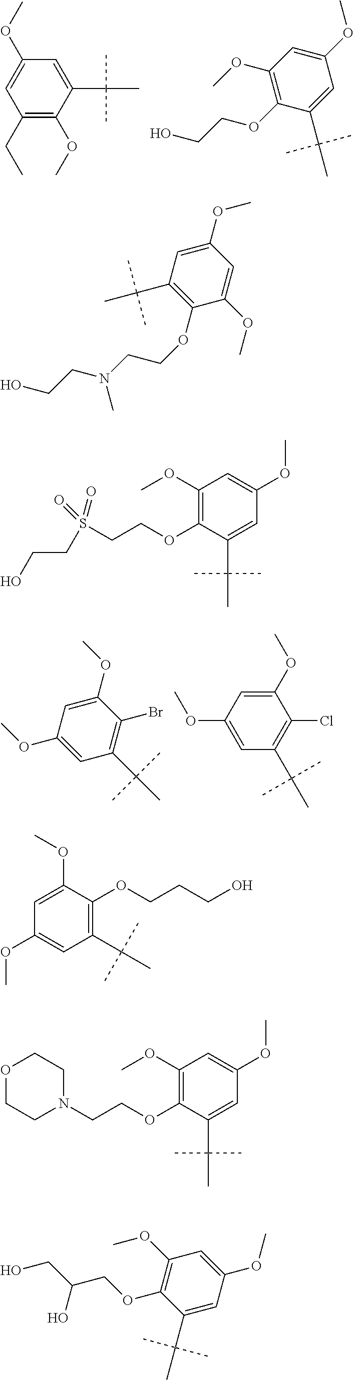

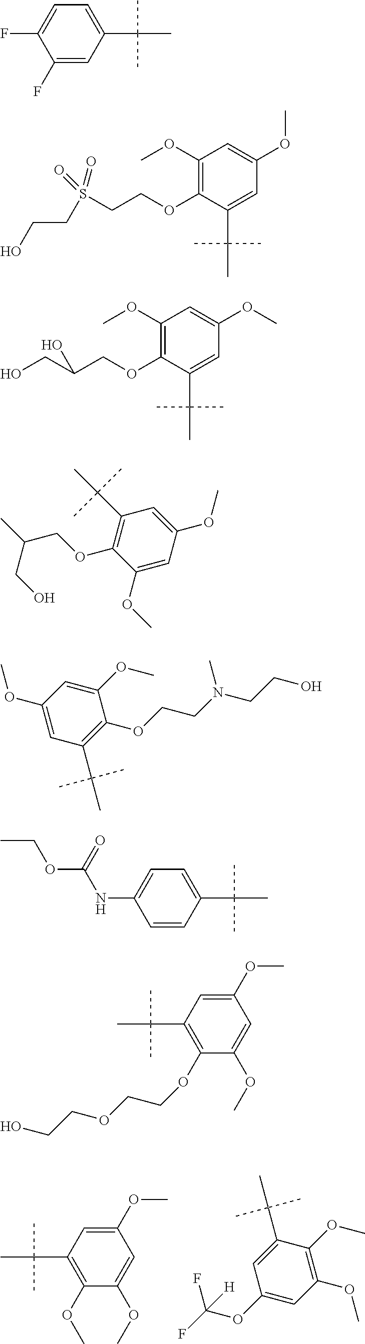

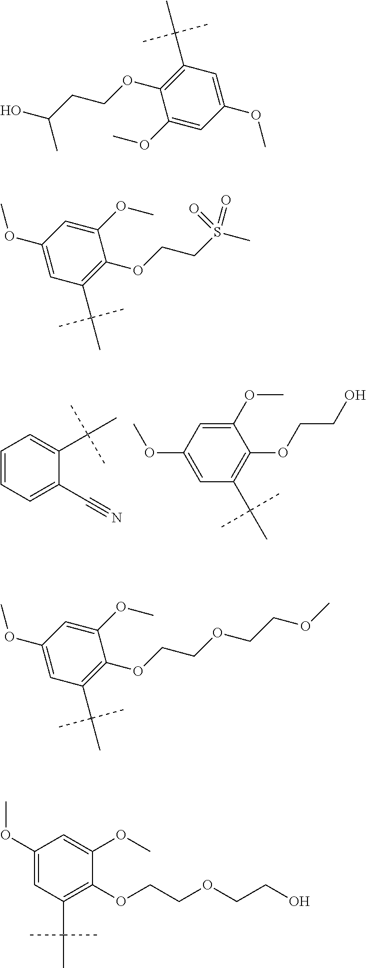

When a variable is present more than one time in a group, each variable independently denotes one of the values provided in its definition.

Het preferably denotes a monocyclic or fused bicyclic saturated, unsaturated or aromatic heterocyclic ring having 1 to 3 N, O and/or S atoms and/or 1 CO group, preferably 1 to 2 N, O and/or S atoms, which may be monosubstituted, disubstituted or trisubstituted by:
Hal,
—C₁-C₆-alkyl, optionally substituted by 1 to 3 Hal, OH, OC₁-C₆-alkyl, —(CH₂—CH₂—O)_{q}CH₃, or —(CH₂—CH₂—O—)_{q}H,
—OC₁-C₆-alkyl optionally substituted by 1 to 3 Hal, OH, OC₁-C₆-alkyl, —(CH₂—CH₂—O)_{q}CH₃, or —(CH₂—CH₂—O—)_{q}H,
CF₃,
OCF₃,
NO₂,
CN,
—(CH₂)ₙNH(C₁-C₆-alkyl),
—(CH₂)ₙCO(C₁-C₆-alkyl),
—(CH2)nCONH(C₁-C₆-alkyl),
—(CH2)nCON(C1-C6-alkyl)2,
—(CH₂)ₙCON(C₁-C₆-alkyl)₂,
—(CH₂)ₙNHCO₂(C₁-C₆-alkyl),
—(CH₂)ₙNHCO(C₁-C₆-alkyl),
—(CH₂)ₙCOHet,
—(CH₂)ₙCOAr,
—(CH₂)ₙN(C₁-C₆-alkyl)(CH₂)ₙAr,
—(CH₂)ₙN(C₁-C₆-alkyl)(CH₂)ₙHet,
n is independently 0, 1, 2 or 3, preferably 0 or 1.
q is independently 0, 1, 2 or 3, preferably 1 or 2.

When Het is a fused bicyclic group, it is enough that one of the cyclic group contains 1 to 4 N, O, and/or S atom or a group selected from CO, SO or SO₂. As examples Het also includes a phenyl or a saturated or unsaturated carbocyclic ring fused with saturated, unsaturated or aro-matic heterocyclic ring having 1 to 4 N, O and/or S atoms and/or 1 group selected from CO, SO or SO₂, and optionally substituted with the substitutents defined in Het.

More preferably, Het denotes one of the following groups:

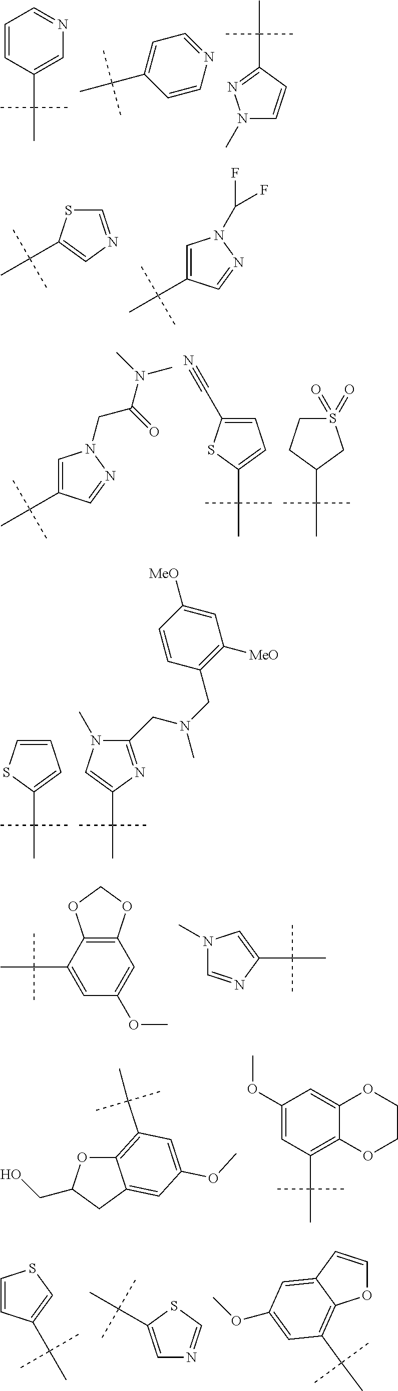

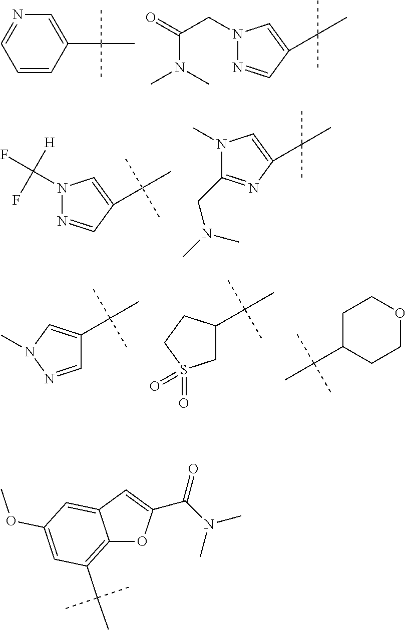

Preferably, the group A denotes a branched or linear alkyl having 1 to 6 C-atoms, wherein one or more, preferably 1 to 3H-atoms may be replaced by:
Hal,
Ar,
Het,
OH,
OC₁-C₆-alkyl optionally substituted by 1 to 3 Hal, OH, OC₁-C₆-alkyl, —(CH₂—CH₂—O)_{q}CH₃, or —(CH₂—CH₂—O—)_{q}H,
CF₃,
OCF₃,
NO₂,
CN,
   and wherein one to 5, preferably 1 to 3 non-adjacent CH₂-groups may be replaced by O, NH, N(C₁-C₆-alkyl) or S.
   The method, according to the present invention, comprises or consists of the following steps 1 and 2:
   Step 1: According to the invention, the intermediate N-(3-chloro-quinoxalin-2-yl)-sulfonamides of formula (II) wherein R¹ is as defined above can be prepared from the 2,3-dichloro-quinoxaline (commercially available or easily obtainable from commercially available starting compounds, scheme 1) by reaction with the sulfonamide of formula (III) wherein R¹ is as above defined, with a alkali metal hydroxide, such as LiOH, KOH or NaOH; preferably LiOH (anhydrous or hydrated form) in an aprotic polar solvent such as DMA, DMSO, DMF or NMP at temperature ranging from 20° C. to 150° C. for period from 0.5 to 48 hours (depending on the nature of the sulfonamide (III)).

Preferably, the reaction is performed in DMA, DMF or DMSO.

Step 2: Then, the intermediate N-(3-chloro-quinoxalin-2-yl)-sulfonamides of formula (II) is transformed into a compound of Formula (I) wherein R¹ is as above defined by reaction with amine of formula NH₂R² where R² is as above defined, using a pyridine base such as pyridine, methylpyridine or dimethylpyridine such as lutidine as a base. The reaction is preferably performed in a polar solvent such as such as DMA, DMF, NMP, DMSO or alcohol (EtOH, MeOH, iPrOH, n-propanol, n-butanol). The temperature of the reaction is ranging from 20° C. to 150° C. for a period from 0.5 to 48 hours (depending on the nature of the amine NH₂R² and of the intermediate (II)).

Preferably, the reaction is performed with 2,6-di-methyl-pyridine (lutidine or 2,6-lutidine) in an alcohol such as n-butanol or n-propanol.

The isolated yield of a compound or an intermediate refers to the yield of such compound or intermediate obtained after a purification step. A purification step is any step intending to remove impurities from the crude mixture after the reaction, using any purification method which is deemed proper. Examples of purification methods are chromatography, crystalisation, distillation, extraction, adsorption, evaporation, centrifugation, or fractionation.

In a first embodiment, the first step of the process of the present invention provides the compounds of Formulae (II) or (II′) in an isolated yield higher than 50%, preferably higher than 70% and most preferably higher than 80%, using an alkali metal hydroxide in a polar aprotic solvent at a temperature between 30° C. and 80° C. More preferably, compounds of Formula (II) are obtained using a polar aprotic solvent at a temperature around 50° C., in a reaction time between 10 hours and 20 hours. More preferably compounds of Formula (II) are obtained in a yield higher than 60% using an alkali metal hydroxide selected from LiOH, KOH and NaOH, preferably LiOH, at a temperature between 40° C. and 60° C., in a polar aprotic solvent selected from DMA, DMSO, NMP, DMF, preferably DMA, in a reaction time of 10 to 24 hours, preferably 15 to 20 hours.

In a second embodiment, the first step of the process of the present invention provides compounds of Formulae (II) or (II′) with a crude purity higher than 70%, using an alkali metal hydroxide in a polar aprotic solvent at a temperature of 40° C. to 60° C. Preferably the reaction time lasts between 10 to 20 hours, more preferably, between 15 to 18 hours. More preferably, the first step of the process of the present invention provides compounds of Formula (II) with a crude purity higher or equal to 80%, using an alkali metal hydroxide selected from LiOH, or KOH, in a polar aprotic solvent selected from DMA, DMSO, NMP and DMF, preferably DMA, at a temperature of 40° C. to 60° C., preferably around 50° C., with a reaction time of 15 to 20 hours, preferably around 16 hours.

In a third embodiment, the first step of the process of the present invention provides compounds of Formulae (II) or (II′) with a crude purity higher than 80%, in a time shorter than 24 hours at a temperature lower than 90° C.

In a fourth embodiment, the first step of the process of the present invention provides compounds of Formulae (II) or (II′) with a crude purity higher than 70%, in a time shorter than 5 hours, preferably shorter than 3 hours, more preferably in around 1 hour, at a temperature lower or equal to 100° C. The polar aprotic solvent is selected from DMF, DMA, NMP and DMSO, preferably DMA. The alkali metal hydroxide is selected from LiOH, KOH and NaOH, preferably LiOH. The amount of alkali metal hydroxide is preferably between 1.8 and 2.5 molar equivalents with respect to 2,3-dichloroquinoxaline, preferably around 2 molar equivalents.

In a fifth embodiment, the first step of the process of the present invention provides compounds of Formulae (II) or (II′) in a crude purity higher than 70% at a temperature lower than 90° C., preferably, in crude purity higher than 70% at a temperature lower than 60° C. The reaction time is preferably between 5 to 24 hours, more preferably between 10 and 20 hours and even more preferably between 15 and 18 hours. The solvent is preferably an aprotic solvent selected from DMF, NMP, DMA, and DMSO, more preferably DMA. The alkali metal base is selected from LiOH, NaOH and KOH, preferably LiOH.

In a sixth embodiment, the alkali metal hydroxide is used in a molar ratio of 0.5 to 2.5 compared to 2,3-dichloroquinoxaline. Preferably, the alkyli metal hydroxide is used in a molar ratio of 0.8 to 1.5 compared to 2,3-dichloroquinoxaline, more preferably in a molar ratio of about 1.2.

In a seventh embodiment, the present invention relates to any compounds of Formulae (I) or (I′) obtained or obtainable by the process described herein.

In a height embodiment, the present invention relates to any compounds of Formulae (II) or (II′) obtained or obtainable by step a) of the process described herein.

### Experimental Part

¹H NMR was recorded on 400 MHz spectrometers. Chemical shifts (6) are reported in ppm relative to the residual solvent signal (δ=2.49 ppm for ¹H NMR in DMSO-d6). ¹H NMR data are reported as follows: chemical shift (multiplicity, coupling constants, and number of hydrogens). Multiplicity is abbreviated as follows: s (singlet), d (doublet), t (triplet), q (quartet), m (multiplet), br (broad).

NMR, HPLC and MS data provided in the examples described below are registered on:
NMR: Bruker DPX-300, using residual signal of deuterated solvent as internal reference.
HPLC: Waters Alliance 2695, column Waters XBridge C8 3.5 μm 4.6×50 mm, conditions: solvent A (H₂O with 0.1% TFA), solvent B (ACN with 0.05% TFA), gradient 5% B to 100% B over 8 min, UV detection with PDA Water 996 (230-400 nm).
LCMS method: 0.1% TFA in H2O, B: 0.1% TFA in ACN Flow Rate: 2.0 mL/min Column: Xbridge C8 (50×4.6 mm, 3.5μ).
UPLC/MS: Waters Acquity, column Waters Acquity UPLC BEH C18 1.7 μm 2.1×50 mm, conditions: solvent A (10 mM ammonium acetate in water+5% ACN), solvent B (ACN), gradient 5% B to 100% B over 3 min, UV detection (PDA, 230-400 nm) and MS detection (SQ detector, positive and negative ESI modes, cone voltage 30V).

### Preparation of N-(3-chloro-quinoxalin-2-yl)-sulfonamides of formula (II)

### Example II-1

### Preparation of N-(3-chloroquinoxalin-2-yl)-1-methyl-1H-imidazole-4-sulfonamide

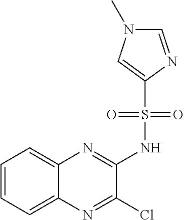 [SMILES: Cn1cnc(S(=O)(=O)Nc2nc3ccccc3nc2Cl)c1]

In a 3 L three-necked round bottom flask containing a solution of 1-methyl-1H-imidazole-4-sulfonamide (80.98 g; 502.4 mmol; 1.0 eq.) in DMA (900 mL), lithium hydroxide (22.86 g; 954.6 mmol; 1.9 eq.) was added in one portion and after stirring for 8 minutes 2,3-dichloroquinoxaline (100 g; 502.4 mmol; 1.0 eq.) was added in one portion.

The reaction mixture was stirred at 50° C. for 16 h until completion (ca about 3% of 2,3-dichloroquinoxaline remaining and only about 1-2% of 3-chloroquinoxalin-2-ol formed as a side product, determined by UPLC/MS). The reaction mixture (yellow solution) was cooled down to 2° C. (ice-bath) and HCl (502.4 mL; 1N) was added drop wise over 40 minutes keeping temperature below 15° C.

Resulting pale yellow fine suspension was stirred for 5 minutes (T=13° C.) and was filtered through a glass filter. The resulting yellow off-white cake was sucked dry under vacuum for 2 h and was then washed twice with cold water (5° C.; 500 mL). The resulting white suspension was sucked dry for 10 minutes and was dried overnight at 40° C. under vacuum to give N-(3-chloroquinoxalin-2-yl)-1-methyl-1H-imidazole-4-sulfonamide (149.32 g, yield, 91.8%, 97% (AUC) by UPLC/MS; 0% of 3-chloroquinoxalin-2-ol; 3% of 2,3-dichloroquinoxaline and by NMR: 2.4% (w/w) of 1-methyl-1H-imidazole-4-sulfonamide as pale yellow powder.

### Example II-2

### Preparation of 2-[(dimethylamino)methyl]-1-methyl-1H-imidazole-4-sulfonamide

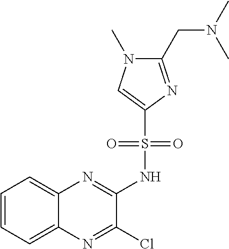 [SMILES: CN(C)Cc1nc(S(=O)(=O)Nc2nc3ccccc3nc2Cl)cn1C]

In a 3 L three-necked round bottom flask containing a solution of 2-[(dimethylamino) methyl]-1-methyl-1H-imidazole-4-sulfonamide (109.67 g; 502.41 mmol; 1.0 eq.) in DMA (900.0 ml), lithium hydroxide (22.86 g; 954.6 mmol; 1.9 eq.) was added in one portion and after stiffing for 8 minutes 2,3-dichloroquinoxaline (100.0 g; 502.41 mmol; 1.0 eq.) was added in one portion. The reaction mixture was stirred at 50° C. for 16 h until completion (ca about 4% of 2,3-dichloroquinoxaline remaining and only 3% of 3-chloroquinoxalin-2-ol formed as a side product, determined by UPLC/MS)

Reaction mixture (brown solution) was cooled down to 5° C. (ice-bath) and HCl (502.4 mL; 1N) was added drop wise over 35 minutes keeping temperature below 17° C. The resulting beige fine suspension was stirred for 5 minutes (T=13° C.) and was filtered through a glass filter. The beige cake was sucked dry under vacuum for 10 minutes and was then washed twice with cold water (T=5° C.; V=2×500 mL; 2×5V). The resulting white suspension was sucked dry over weekend and was dried for 16 h at 40° C. under 30 mbar to give N-(3-chloroquinoxalin-2-yl)-2-[(dimethylamino)methyl]-1-methyl-1H-imidazole-4-sulfonamide [168.44 g, yield, 88%, 94% (AUC) by UPLC/MS; 1.25% of 3-chloroquinoxalin-2-ol; 3.4% of 2,3-dichloroquinoxaline and by NMR: 3.6% (w/w) of 2-[(dimethylamino)methyl]-1-methyl-1H-imidazole-4-sulfonamide as off-white powder.

### Example II-4

### Preparation of N-(3-chloroquinoxalin-2-yl)-4-fluorobenzenesulfonamide

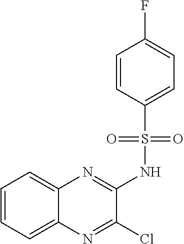 [SMILES: O=S(=O)(Nc1nc2ccccc2nc1Cl)c1ccc(F)cc1]

In a 150 mL flask under nitrogen containing a solution of 4-fluorobenzenesulfonamide (4.40 g; 25.12 mmol; 1.0 eq.) in DMA (45.00 ml), lithium hydroxide (1.14 g; 47.73 mmol; 1.9 eq.) was added in one portion and after stiffing for 10 minutes 2,3-dichloroquinoxaline (5.00 g; 25.12 mmol; 1.0 eq.) was added in one portion. Reaction mixture was stirred at 50° C. for 20 h until completion as indicated by UPLC/MS The reaction mixture (yellow solution) was cooled down to 5° C. (ice-bath) and HCl (25.12 ml; 1N) was added in one pot and resulting suspension was aged in an ice bath for 20 minutes until complete precipitation. Then suspension was filtered and washed with water (3×50 mL), then resulting solid was washed with MTBE to remove 2, 3 dichloroquinoxaline in excess (2×30 mL). Additional crop was obtained upon precipitation in the MTBE phase (heptane was added to the filtrate to initiate precipitation and a second crop was obtained by filtration). The 2 crops were combined to give after drying title product as white powder (5.59 g; 65.9%).

HPLC Purity: 99.3% (max plot), Rt: 3.76 min; UPLC/MS: purity: 100% (max plot), Rt: 1.06 min

### Example II-5 (using lithium hydroxide as a base)

### Preparation of 2-chloro-N-(3-chloroquinoxalin-2-yl) benzene sulfonamide

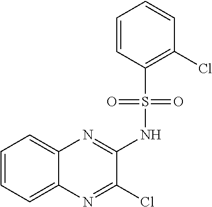 [SMILES: O=S(=O)(Nc1nc2ccccc2nc1Cl)c1ccccc1Cl]

In a 150 mL flask under N₂ containing a solution of 2-chlorobenzenesulphonamide (4.81 g; 25.1 mmol; 1.0 eq.) in DMA (45 mL), lithium hydroxide (1.14 g; 47.7 mmol; 1.9 eq.) was added in one portion and after stirring for 10 minutes 2,3-dichloroquinoxaline (5.0 g; 25.12 mmol; 1.0 eq.) was added in one portion. The reaction mixture was stirred at 50° C. for 20 h until completion as indicated by UPLC/MS.

The reaction mixture (clear brown solution) was then cooled down to 5° C. (ice-bath) and hydrochloric acid 1N (25.1 mL) was added in one pot. The resulting suspension was aged in a ice bath for 20 minutes until complete precipitation. Then, the suspension was filtered and washed with water (3×50 mL), and the resulting solid was washed with MTBE (2×30 mL) to remove 2, 3 dichloroquinoxaline in excess. Additional crop was obtained upon precipitation in the MTBE phase (heptane was added to the filtrate to initiate precipitation and a second crop was obtained by filtration). The 2 crops were combined to give after drying title product as beige solid (6.25 g; crude yield: 70.2%).

HPLC Purity: 98.9% (max plot), Rt: 3.86 min; UPLC/MS: purity 100% (max plot), Rt: 1.08 min

### Example II-5 (using potassium carbonate as a base)

### Preparation of 2-chloro-N-(3-chloroquinoxalin-2-yl) benzene sulfonamide

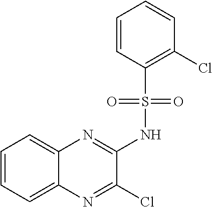 [SMILES: O=S(=O)(Nc1nc2ccccc2nc1Cl)c1ccccc1Cl]

In 25 mL flask under N₂ containing a solution of 2-chlorobenzenesulphonamide (0.48 g; 2.51 mmol; 1.0 eq.) in DMA (4.5 mL), potassium carbonate (0.66 g; 4.77 mmol; 1.9 eq.) was added in one portion and after stirring for 10 minutes 2,3-dichloroquinoxaline (500 mg; 2.51 mmol; 1.0 eq.) was added in one portion. The reaction mixture was stirred at 50° C. for 22 h until analysis by UPLC/MS. The reaction mixture (yellow solution) was then stirred at 100° C. for the weekend until analysis by UPLC/MS. The reaction mixture was then cooled down to 5° C. (ice-bath) and hydrochloric acid 1N (5.0 mL) was added in one pot and resulting suspension was filtered and washed with water then with MTBE to give after drying title product as beige solid (173 mg; crude yield: 19.4%). PLC/MS: purity: 100% (max plot), Rt: 1.08 min

The following further compounds may be obtained using the above set of protocols using lithium, potassium hydroxide or alkali metal hydroxide (Table 1).

TABLE 1
Example II-3 to Example II-30

| | |
|---|---|
| 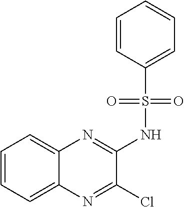 [SMILES: O=S(=O)(Nc1nc2ccccc2nc1Cl)c1ccccc1] | II-3 |
| 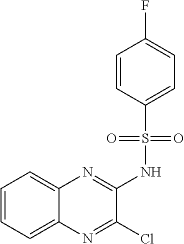 [SMILES: O=S(=O)(Nc1nc2ccccc2nc1Cl)c1ccc(F)cc1] | II-4 |
| 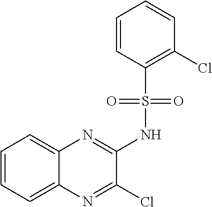 [SMILES: O=S(=O)(Nc1nc2ccccc2nc1Cl)c1ccccc1Cl] | II-5 |
| 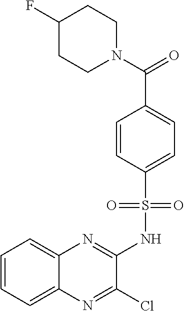 [SMILES: O=C(c1ccc(S(=O)(=O)Nc2nc3ccccc3nc2Cl)cc1)N1CCC(F)CC1] | II-6 |
| 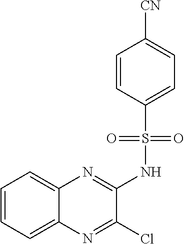 [SMILES: N#Cc1ccc(S(=O)(=O)Nc2nc3ccccc3nc2Cl)cc1] | II-7 |
| 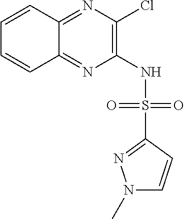 [SMILES: Cn1ccc(S(=O)(=O)Nc2nc3ccccc3nc2Cl)n1] | II-8 |
| 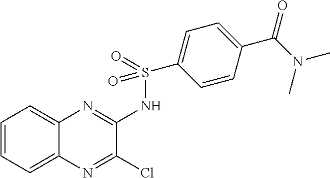 [SMILES: CN(C)C(=O)c1ccc(S(=O)(=O)Nc2nc3ccccc3nc2Cl)cc1] | II-9 |
| 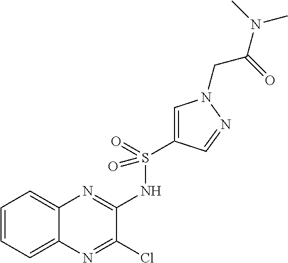 [SMILES: CN(C)C(=O)Cn1cc(S(=O)(=O)Nc2nc3ccccc3nc2Cl)cn1] | II-10 |
| 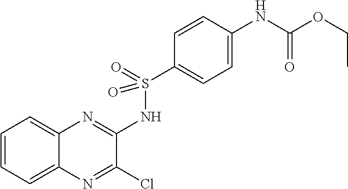 [SMILES: CCOC(=O)Nc1ccc(S(=O)(=O)Nc2nc3ccccc3nc2Cl)cc1] | II-11 |
| 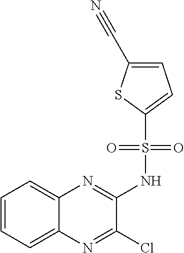 [SMILES: N#Cc1ccc(S(=O)(=O)Nc2nc3ccccc3nc2Cl)s1] | II-12 |
| 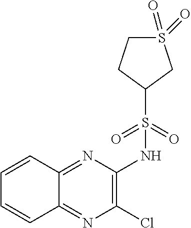 [SMILES: O=S1(=O)CCC(S(=O)(=O)Nc2nc3ccccc3nc2Cl)C1] | II-13 |
| 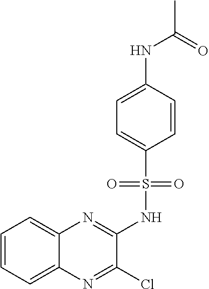 [SMILES: CC(=O)Nc1ccc(S(=O)(=O)Nc2nc3ccccc3nc2Cl)cc1] | II-14 |
| 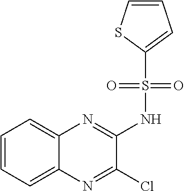 [SMILES: O=S(=O)(Nc1nc2ccccc2nc1Cl)c1cccs1] | II-15 |
| 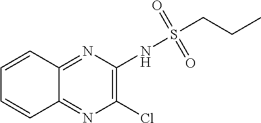 [SMILES: CCCS(=O)(=O)Nc1nc2ccccc2nc1Cl] | II-16 |
| 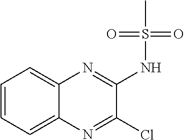 [SMILES: CS(=O)(=O)Nc1nc2ccccc2nc1Cl] | II-18 |
| 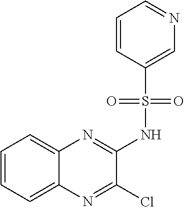 [SMILES: O=S(=O)(Nc1nc2ccccc2nc1Cl)c1cccnc1] | II-19 |
| 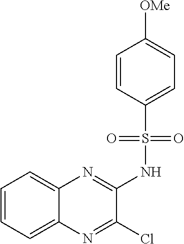 [SMILES: COc1ccc(S(=O)(=O)Nc2nc3ccccc3nc2Cl)cc1] | II-20 |
| 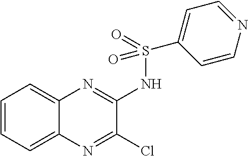 [SMILES: O=S(=O)(Nc1nc2ccccc2nc1Cl)c1ccncc1] | II-21 |
| 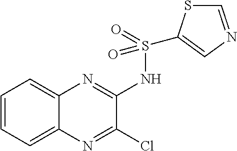 [SMILES: O=S(=O)(Nc1nc2ccccc2nc1Cl)c1cncs1] | II-22 |
| 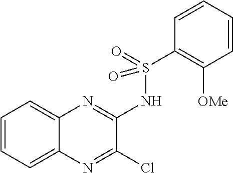 [SMILES: COc1ccccc1S(=O)(=O)Nc1nc2ccccc2nc1Cl] | II-23 |
| 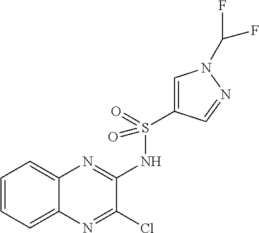 [SMILES: O=S(=O)(Nc1nc2ccccc2nc1Cl)c1cnn(C(F)F)c1] | II-24 |
| 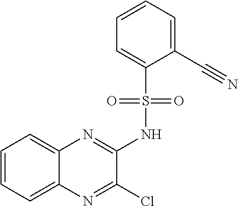 [SMILES: N#Cc1ccccc1S(=O)(=O)Nc1nc2ccccc2nc1Cl] | II-25 |
| 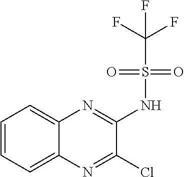 [SMILES: O=S(=O)(Nc1nc2ccccc2nc1Cl)C(F)(F)F] | II-26 |
| 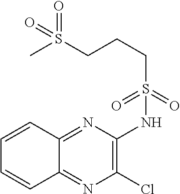 [SMILES: CS(=O)(=O)CCCS(=O)(=O)Nc1nc2ccccc2nc1Cl] | II-27 |
| 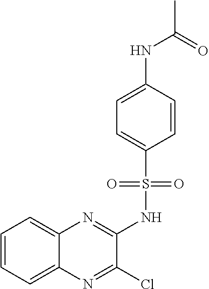 [SMILES: CC(=O)Nc1ccc(S(=O)(=O)Nc2nc3ccccc3nc2Cl)cc1] | II-28 |
| 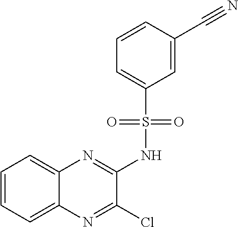 [SMILES: N#Cc1cccc(S(=O)(=O)Nc2nc3ccccc3nc2Cl)c1] | II-29 |
| 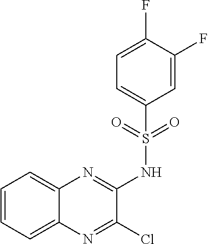 [SMILES: O=S(=O)(Nc1nc2ccccc2nc1Cl)c1ccc(F)c(F)c1] | II-30 |
| 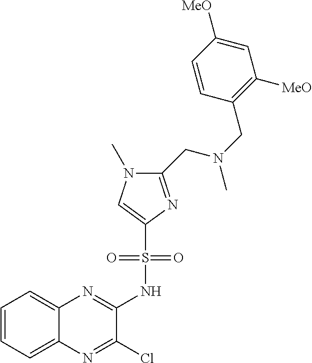 [SMILES: COc1ccc(CN(C)Cc2nc(S(=O)(=O)Nc3nc4ccccc4nc3Cl)cn2C)c(OC)c1] | II-17 |

The preferred conditions are the ones using lithium hydroxide in DMA at a temperature of about 50° C. Better isolated yields were obtained using lithium hydroxide over other bases, such as K₂CO₃ (Table 2).

TABLE 2
Comparative isolated yield following the use of LiOH or K₂CO₃
as a base for the reaction of the 2,3-dichloro-quinoxaline with the
sulfonamide of formula (III) where R¹ is selected from the
group consisting of alkyl, cylcoalkyl, heterocycloalkyls, aryl and
heteroaryl

Conditions

| | LiOH, DMA, 50° C. | K₂CO₃, DMA, 50° C. |
|---|---|---|
| Examples | 18 h | 18 h |
| II-3 | 67% | 20% |
| II-4 | 66% | 51% |
| II-5 | 70% | 19% |
| II-6 | 76% | — |
| II-7 | 73% | 40% |
| II-18 | 52% | 4% |
| II-19 | 51% | — |
| II-20 | 73% | 18% |
| II-1 | 90% | — |
| II-2 | 88% | — |

In addition, when an alkali metal hydroxide is used, the purity profile of the crude reaction is improved (Table 3). The formation of the byproduct or impurity during the reaction minimized in these conditions as compared to the use of other bases such as K₂CO₃.

The purity profiles of the crude reaction depicted in Table 3 are determined by chromatography analysis with the following HPLC Method: Solvant A: 0.1% TFA in H₂O, Solvent B: 0.1% TFA in ACN: Flow-2.0 mL/min. Column: Waters X Bridge C8 (50×4.6 mm, 3.50.

In particular, the formation of impurity or byproduct E during the reaction is minimized when LiOH is used as compared to other bases such as K₂CO₃. Removal of the impurity or byproduct E from the desired N-(3-chloro-quinoxalin-2-yl)-sulfonamides of formula (II) often required extensive washing, crystallization or other purification procedures.

TABLE 3
Table of ratio of product, reactant and impurities (determined by
UPLC/MS and expressed in %) following the reaction of the 2,3-
dichloro-quinoxaline with the sulfonamide of formula (III)
wherein R¹ is as defined above, to give the intermediate (II)

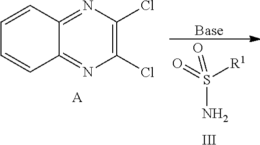

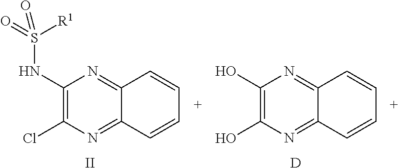

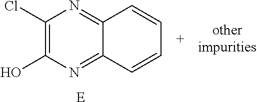

| | Impurities or | Conditions | | | |
|---|---|---|---|---|---|
| Examples | compound | C1 | C2 | C3 | C4 |
| II-1 | A | 3 | nd | nd | 3.8 |
| | III | 0 | nd | nd | bdl |
| | II | 93 | nd | nd | 62 |
| | D | 2.3 | nd | nd | 2.18 |
| | E | 1.47 | nd | nd | 2.75 |
| | Other | 0.23 | nd | nd | 29.27 |
| | byproduct(s) | | | | |
| II-2 | A | 4 | nd | nd | bdl |
| | III | 0 | nd | nd | bdl |
| | II | 90 | nd | nd | 66 |
| | D | 2.54 | nd | nd | 12 |
| | E | 3.03 | nd | nd | 13 |
| | Other | 0.43 | nd | nd | 9 |
| | byproduct(s) | | | | |
| II-3 | A | 6 | 5 | 46 | bdl |
| | III | bdl | bdl | 2 | bdl |
| | II | 91 | 89 | 48 | 89 |
| | D | 2.7 | 2.4 | 3.4 | 2.7 |
| | E | bdl | 3.6 | bdl | 3.6 |
| | Other | 0.3 | bdl | 0.6 | 4.7 |
| | byproduct(s) | | | | |
| II-4 | A | 7 | nd | nd | nd |
| | III | bdl | nd | nd | nd |
| | C | 90 | nd | nd | nd |
| | D | 2.8 | nd | nd | nd |
| | E | bdl | nd | nd | nd |
| | Other | 0.2 | nd | nd | nd |
| | byproduct(s) | | | | |
| II-5 | A | 6 | 6 | 22 | bdl |
| | III | bdl | bdl | 2 | bdl |
| | II | 91 | 89 | 73 | 98 |
| | D | 2.8 | 2.5 | 3.3 | 1.92 |
| | E | bdl | 2.6 | bdl | bdl |
| | Other | 0.2 | bdl | bdl | 0.08 |
| | byproduct(s) | | | | |
| II-6 | A | 7 | nd | nd | nd |
| | III | 2.6 | nd | nd | nd |
| | II | 87 | nd | nd | nd |
| | D | 2.1 | nd | nd | nd |
| | E | bdl | nd | nd | nd |
| | Other | 1.3 | nd | nd | nd |
| | byproduct(s) | | | | |
| II-7 | A | 6 | 6 | 18 | bdl |
| | III | 4 | bdl | 11 | bdl |
| | II | 88 | 89 | 69 | 92 |
| | D | 2.6 | 2.5 | 2.4 | 2.09 |
| | E | bdl | 2.6 | bdl | bdl |
| | Other | bdl | bdl | bdl | 5.91 |
| | byproduct(s) | | | | |
| II-8 | A | 8 | nd | nd | nd |
| | III | 3 | nd | nd | nd |
| | II | 90 | nd | nd | nd |
| | D | 2.9 | nd | nd | nd |
| | E | bdl | nd | nd | nd |
| | Other | bdl | nd | nd | nd |
| | byproduct(s) | | | | |
| II-9 | A | 25 | 14 | 34 | bdl |
| | III | bdl | bdl | 17 | bdl |
| | II | 71 | 81 | 45 | 70 |
| | D | 2.95 | 3.5 | 2.18 | 5 |
| | E | bdl | bdl | bdl | 15.7 |
| | Other | 1.05 | 1.5 | bdl | 9.3 |
| | byproduct(s) | | | | |
| II-18 | A | 7 | 6 | 40 | bdl |
| | III | bdl | bdl | bdl | bdl |
| | II | 90 | 88 | 54 | 97 |
| | D | 2.4 | 2.8 | 2.9 | bdl |
| | E | 1.02 | 3.3 | 1 | 1.8 |
| | Other | bdl | bdl | 2.1 | 1.2 |
| | byproduct(s) | | | | |
| II-20 | A | 7 | 6 | 34 | bdl |
| | III | 4 | 5 | 17 | 1.35 |
| | II | 86 | 83 | 45 | 92 |
| | D | 2.2 | 2.3 | 2.18 | bdl |
| | E | 1.1 | 3.6 | bdl | 2.4 |
| | Other | bdl | 0.1 | 1.82 | 4.25 |
| | byproduct(s) | | | | |

Conditions:
C1: LiOH (DMA, 16 h, 50° C.),
C2: KOH (DMA, 16 h, 50° C.);
C3: K₂CO₃ (DMSO, 16 h, 50° C.);
C4: K₂CO₃ (DMA, 48 h, 100° C.)
Ratio of compounds are measured by UPLC/MS: Waters Acquity, column Waters Acquity UPLC BEH C18 1.7 μm 2.1 × 50 mm, conditions: solvent A (10 mM ammonium acetate in water + 5% ACN), solvent B (ACN), gradient 5% B to 100% B over 3 min, UV detection (PDA, 230-400 nm) and MS detection (SQ detector, positive and negative ESI modes, cone voltage 30 V).
bdl: below detection limit (UPLC/MS)
nd: not determined

Other examples of bases for the reaction are exemplified in the table 4, illustrating the improved purity profile from the reaction mixture, and the use of lower reaction temperature to reach similar or better isolated yields (Table 4).

TABLE 4
Comparative purity profile (determined by UPLC/MS) of crude reaction mixture
following the above protocol described for the preparation of Example II-1 with different
bases under different conditions starting, from 1 eq. of 2,3-dichloroquinoxaline A and 1
or 1.05 eq. of 1-methyl-1H-imidazole-4-sulfonamide of formula (III).

| | | | | | | | | | Crude |
|---|---|---|---|---|---|---|---|---|---|
| | | | | | | | | | Purity |
| # | Base | | | | | | | Isolated | (measured |
| Equiv. | | # | Solvent | | | | | Yield | by |
| III | Name | Equiv. | Name | Volume | Temp. | | Time | (%) | UPLC/MS) |
| 1 | 2,6-lutidine | 1.1 | 1-butanol | 20 | 120° | C. | 20 h | — | (1) |
| 1 | 2,6-lutidine | 1.1 | DMF | 20 | 120° | C. | 20 h | — | (1) |
| 1 | no | | DMF | 20 | 120° | C. | 20 h | — | (2) |
| 1 | no | | 1-butanol | 20 | 120° | C. | 20 h | — | (2) |
| 1 | K₂CO₃ | 1 | DMF | 20 | 120° | C. | 20 h | — | 74% |
| 1 | K₂CO₃ | 1 | 1-butanol | 20 | 120° | C. | 20 h | — | 34% |
| 1 | K₂CO₃ | 1 | DMF | 20 | 120° | C. | 20 h | 59 | 93% |
| 1 | Cs₂CO₃ | 1 | DMF | 20 | 100° | C. | 23 h | 48 | 84% |
| 1 | Cs₂CO₃ | 1 | DMSO | 8 | 90° | C. | 19 h | — | 57% |
| 1 | K₂CO₃ | 1 | DMF | 12 | 100° | C. | 16 h | — | 62% |
| 1 | K₂CO₃ | 1 | NMP | 12 | 100° | C. | 16 h | — | 60% |
| 1 | K₂CO₃ | 1 | DMSO | 12 | 100° | C. | 16 h | — | 77% |
| 1 | K₂CO₃ | 1 | DMA | 12 | 100° | C. | 16 h | — | 62% |
| 1 | K₂CO₃ | 1 | 2-methyl-2- | 12 | 100° | C. | 16 h | — | 3% |
| | | | propanol | | | | | | |
| 1 | Cs₂CO₃ | 1 | DMF | 12 | 100° | C. | 16 h | — | 77% |
| 1 | Cs₂CO₃ | 1 | NMP | 12 | 100° | C. | 16 h | — | 66% |
| 1 | Cs₂CO₃ | 1 | DMSO | 12 | 100° | C. | 16 h | — | 82% |
| 1 | Cs₂CO₃ | 1 | DMA | 12 | 100° | C. | 16 h | — | 70% |
| 1 | Cs₂CO₃ | 1 | 2-methyl-2- | 12 | 100° | C. | 16 h | — | 4% |
| | | | propanol | | | | | | |
| 1 | Na₂CO₃ | 1 | DMF | 12 | 100° | C. | 16 h | — | 30% |
| 1 | Na₂CO₃ | 1 | NMP | 12 | 100° | C. | 16 h | — | 34% |
| 1 | Na₂CO₃ | 1 | DMSO | 12 | 100° | C. | 16 h | — | 58% |
| 1 | Na₂CO₃ | 1 | DMA | 12 | 100° | C. | 16 h | — | 35% |
| 1 | Na₂CO₃ | 1 | 2-methyl-2- | 12 | 100° | C. | 16 h | — | 0% |
| | | | propanol | | | | | | |
| 1 | LiOH | 2.5 | DMA | 10 | 50° | C. | 6 h 30 | 79 | 86% |
| 1 | LiOH | 1.25 | DMA | 10 | 50° | C. | 24 h | — | 71% |
| 1 | LiOH | 1.5 | DMA | 10 | 50° | C. | 24 h | — | 80% |
| 1 | LiOH | 1.87 | DMA | 10 | 50° | C. | 24 h | — | 93% |
| 1 | LiOH | 2 | DMA | 10 | 50° | C. | 3 h | — | 93% |
| 1.05 | LiOH | 1.5 | DMA | 10 | 50° | C. | 2 h | — | 81% |
| 1.05 | LiOH | 1.9 | DMA | 10 | 50° | C. | 2 h | — | 91% |
| 1 | LiOH | 2 | DCM | 10 | 40° | C. | 16 h | — | bdl |
| 1 | LiOH | 2 | THF | 10 | 40° | C. | 16 h | — | bdl |
| 1 | LiOH | 2 | DMA | 10 | 40° | C. | 16 h | — | 81% |
| 1 | LiOH | 2 | dioxane | 10 | 40° | C. | 16 h | — | bdl |
| 1 | LiOH | 2 | EtOH | 10 | 40° | C. | 16 h | — | bdl |
| 1 | LiOH | 2 | DMSO | 10 | 40° | C. | 16 h | — | 98% |
| 1 | LiOH | 2 | iPrOH | 10 | 40° | C. | 16 h | — | bdl |
| 1 | LiOH | 2 | iPrOEt | 10 | 40° | C. | 16 h | — | bdl |
| 1 | LiOH | 2 | CHCI3 | 10 | 40° | C. | 16 h | — | bdl |
| 1 | LiOH | 2 | Toluene | 10 | 40° | C. | 16 h | — | bdl |
| 1 | LiOH | 2 | CH3CN | 10 | 40° | C. | 16 h | — | bdl |
| 1 | LiOH | 2 | Acetone | 10 | 40° | C. | 16 h | — | bdl |
| 1 | LiOH | 2 | DCE | 10 | 40° | C. | 16 h | — | bdl |
| 1 | LiOH | 2 | n-Butanol | 10 | 40° | C. | 16 h | — | bdl |
| 1 | LiOH | 2 | 2-butanol | 10 | 40° | C. | 16 h | — | bdl |
| 1 | LiOH | 2 | NMP | 10 | 40° | C. | 16 h | — | 90% |
| 1 | LiOH | 2 | DMF | 10 | 40° | C. | 16 h | — | 92% |
| 1 | LiOH | 2 | Ethyl acetate | 10 | 40° | C. | 16 h | — | bdl |
| 1.05 | LiOH | 2.1 | DMSO | 2 | 50° | C. | 2 h 30 | — | 78% |
| 1.05 | LiOH | 2.5 | DMSO | 2 | 50° | C. | 2 h 30 | — | 77% |
| 1 | LiOH | 2 | DMA | 5 | 50° | C. | 1 h | — | 78% |
| 1 | LiOH | 2 | DMA | 10 | 50° | C. | 1 h | — | 90% |
| 1 | LiOH | 2 | DMA | 10 | 100° | C. | 1 h | — | 92% |
| 1 | LiOH | 1.9 | DMA | 7 | 50° | C. | 17 h | — | 87% |
| 1 | LiOH | 1.9 | DMA | 8 | 50° | C. | 17 h | — | 87% |
| 1 | LiOH | 1.9 | DMA | 9 | 50° | C. | 14 h | 90 | 94% |

The purity of N-(3-chloroquinoxalin-2-yl)-1-methyl-1H-imidazole-4-sulfonamide II-1 was determined by UPLC/MS of the crude mixture using the following method: Waters Acquity, column Waters Acquity UPLC BEH C18 1.7 μm 2.1 × 50 mm, conditions: solvent A (10 mM ammonium acetate in water +5% ACN), solvent B (ACN), gradient 5% B to 100% B over 3 min, UV detection (PDA, 230-400 nm) and MS detection (SQ detector, positive and negative ESI modes, cone voltage 30 V).
bdl: below detection limit (UPLC/MS)
(1) Formation of major Impurity D
(2) Very low conversion

The synthesis of compounds of Formula (II′) is illustrated by the following example wherein A′ is reacted with B′. The crude ratio of compounds A′, B′ C′ and D′, in table 5, has been determined according to the method described above.

TABLE 5

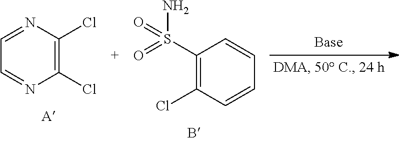 [SMILES: Clc1nccnc1Cl ; NS(=O)(=O)c1ccccc1Cl]

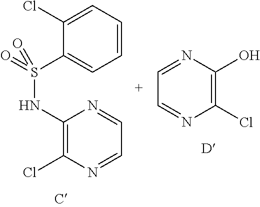

| | base | A′ (%) | B′ (%) | C′ (%) | D′ (%) |
|---|---|---|---|---|---|
| Retention time (min) | | 1.54 | 1.02 | 1.19 | 0.27 |
| Experiment 1 | K₂CO₃ | 44 | 9 | 43 | 3 |
| Experiment 2 | KOH | 22 | 7 | 44 | 27 |
| Experiment 3 | LiOH | 15 | 4 | 76 | 6 |

### Preparation of N-(3-amino-quinoxalin-2-yl)-sulfonamides of formula (I)

In a second step, the intermediate N-(3-chloro-quinoxalin-2-yl)-sulfonamides of formula (II) is converted to the N-(3-amino-quinoxalin-2-yl)-sulfonamides (I) by reaction with an amine of formula NH₂R² wherein R² is as above defined (Scheme 1, Step 2) with a pyridine base, preferably 2,6-dimethylpyridine (lutidine). Preferably, the amount of the pyridine base i.e. lutidine, is between 0.5 and 2 molar equivalent compared to compounds of Formula (II), more preferably between 0.8 and 1.2 molar equivalents, more preferably around 1.1 molar equivalent.

### Example I-1

### N-(3-{[2-(3-hydroxypropoxy)-3,5-dimethoxyphenyl]-amino}quinoxalin-2-yl)-1-methyl-1H-pyrazole-3-sulfonamide

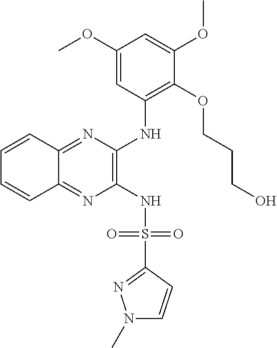 [SMILES: COc1cc(Nc2nc3ccccc3nc2NS(=O)(=O)c2ccn(C)n2)c(OCCCO)c(OC)c1]

200 mg of N-(3-chloroquinoxalin-2-yl)-1-methyl-1H-pyrazole-3-sulfonamide, 180 mg of 3-(2-amino-4,6-dimethoxyphenoxy)propan-1-ol and 72 μL of Lutidine were poured into 2 mL of propanol and heated at 140° C. under microwave irradiation (high absorption mode) for ca. 3 h until completion of reaction. The reaction mixture was cooled to RT, filtered and the collected product was washed with 1-propanol and then dried under vacuum. 251 mg of N-(3-{[2-(3-hydroxypropoxy)-3,5-dimethoxyphenyl]amino}quinoxalin-2-yl)-1-methyl-1H-pyrazole-3-sulfonamide was isolated as a light yellow powder (80%). MS-FAB (M+H⁺)=515.1.

### Example I-2

### Preparation of N-(3-{[2-(3-hydroxypropyl)-5-methoxyphenyl]-amino}quinoxalin-2-yl)-1-methyl-1H-imidazole-4-sulfonamide

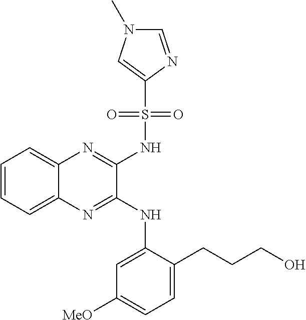 [SMILES: COc1ccc(CCCO)c(Nc2nc3ccccc3nc2NS(=O)(=O)c2cn(C)cn2)c1]

In a 4 L three necked-flask under N₂, containing N-(3-chloroquinoxalin-2-yl)-1-methyl-1H-imidazole-4-sulfonamide (100.0 g; 308.9 mmol; 1.0 eq.) and 3-(2-amino-4-methoxy-phenyl)-propan-1-ol (61.58 g; 339.8 mmol; 1.1 eq.) suspended in 1-butanol (2 L), 2,6-dimethylpyridine (39.44 mL; 339.8 mmol; 1.1 eq.) was added in one portion.

The reaction mixture was stirred at 120° C. (oil bath at 125° C.) under N₂ for 42 h until completion of reaction.

The temperature was allowed to cool down to RT and the reaction mixture was filtered through a glass filter and resulting yellow cake was washed first twice with n-butanol (2×400 ml) then twice with distilled water (2×500 mL). After filtration and sucking dry for 30 minutes, 100% purity was achieved (determined by UPLC/MS). The product was dried under vacuum at 35° C. for 2 days until no weight variation was observed anymore to give N-(3-{[2-(3-hydroxypropyl)-5-methoxyphenyl]amino}quinoxalin-2-yl)-1-methyl-1H-imidazole-4-sulfonamide [115.69 g, yield: 79.9%, 98% (AUC) by HPLC; CHN: [C22H24N6O4S] Corrected: C56.40%, H5.16%, N17.94%;

Found: C56.30%, H5.12%, N17.86%; 0.1% Cl; <0.1% water; 0.3% n-butanol by NMR.]

Other examples of bases for the above reaction are exemplified in the table 6, illustrating the improved purity profile by the use of lutidine.

TABLE 6
Comparative purity profile (determined by UPLC/MS) of crude reaction mixture
following the above protocol described for the preparation of Example I-2 with different bases
under different conditions starting, from N-(3-chloroquinoxalin-2-yl)-1-methyl-1H-imidazole-4-
sulfonamide B and 3-(2-amino-4-methoxyphenyl)propan-1-ol A. Better crude purities
(determined by UPLC/MS) were obtained using lutidine over other bases, such as pyridine,
DMAP, N-methyl-imidazole.

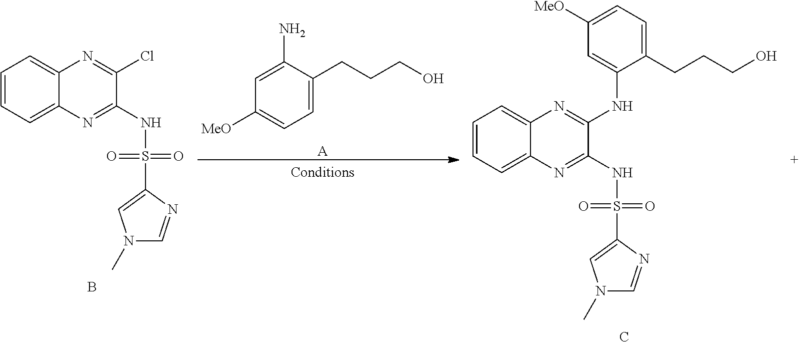

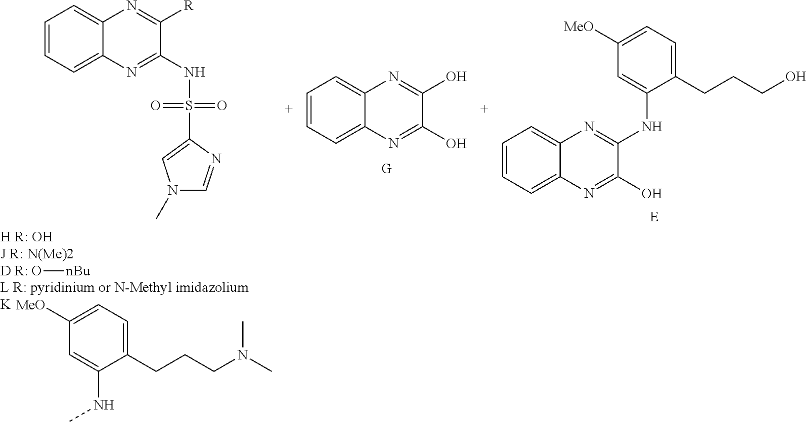

| | A | B | C | D | E | F | G | H | I | J | K | L | |
|---|---|---|---|---|---|---|---|---|---|---|---|---|---|
| Conditions | (%) | (%) | (%) | (%) | (%) | (%) | (%) | (%) | (%) | (%) | (%) | (%) | Others* |
| 1.1 eq. A, 1.1 eq. | — | 31 | 41 | — | — | — | 4 | 16 | — | 7 | — | — | — |
| Pyridine, DMA (20 | | | | | | | | | | | | | |
| V), 100° C. 17 h | | | | | | | | | | | | | |
| 1.1 eq. A, 1.1 eq. | — | 25 | 50 | — | — | — | 5 | 16 | — | — | — | — | 4 |
| Pyridine, NMP (20 | | | | | | | | | | | | | |
| V), 100° C. 17 h | | | | | | | | | | | | | |
| 1.1 eq. A, 1.1 eq. | 12 | 26 | 52 | — | 4 | — | 6 | — | — | — | — | — | — |
| Pyridine, Butyl | | | | | | | | | | | | | |
| ether (20 V), | | | | | | | | | | | | | |
| 100° C. 17 h | | | | | | | | | | | | | |
| 1.1 eq. A, 1.1 eq. | — | — | — | — | — | — | — | 56 | — | — | — | 19 | 25 |
| N-methyl | | | | | | | | | | | | | |
| Imidazole, 120° C. | | | | | | | | | | | | | |
| 17 h | | | | | | | | | | | | | |
| 1.1 eq. A, 1.1 eq. | 15 | — | 73 | | 4 | — | — | — | — | — | — | — | 8 |
| N-methyl | | | | | | | | | | | | | |
| Imidazole, n- | | | | | | | | | | | | | |
| butanol (20 V), | | | | | | | | | | | | | |
| 100° C. 17 h | | | | | | | | | | | | | |
| 1.3 eq. A, 1.0. eq. | — | — | 59 | | 7 | — | 7 | — | — | — | — | — | 27 |
| N,N-dimetyl | | | | | | | | | | | | | |
| aniline, n-butanol | | | | | | | | | | | | | |
| (5 V), 100° C. 16 h | | | | | | | | | | | | | |
| 1.1 eq. A, 1.1 eq. | 2 | — | 86 | — | — | — | — | — | — | — | — | — | 12 |
| Lutidine, n- | | | | | | | | | | | | | |
| butanol, (5 V), | | | | | | | | | | | | | |
| 120° C. | | | | | | | | | | | | | |

Purity profile within the crude mixture has been measured by UPLC/MS using the following method: Waters Acquity, column Waters Acquity UPLC BEH C18 1.7 μm 2.1x50 mm, conditions: solvent A (10 mM ammonium acetate in water + 5% ACN), solvent B (ACN), gradient 5% B to 100% B over 3 min, UV detection (PDA, 230-400 nm) and MS detection (SQ detector, positive and negative ESI modes, cone voltage 30 V).
*Other refer to ratio of uncharacterized compounds, following UPLC/MS analysis of the crude reaction mixture.
NA: not applicable

The following further compounds I-3 to I-93 may be obtained using the above set out protocols (Table 7), in particular using lutidine as a base.

TABLE 7

| Ex. | Structure |
|---|---|
| I-3 | 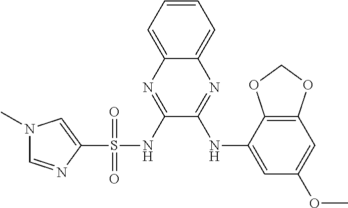 |
| I-4 | 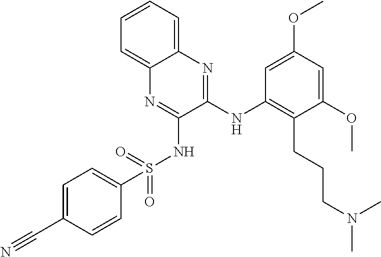 [SMILES: COc1cc(Nc2nc3ccccc3nc2NS(=O)(=O)c2ccc(C#N)cc2)c(CCCN(C)C)c(OC)c1] |
| I-5 | 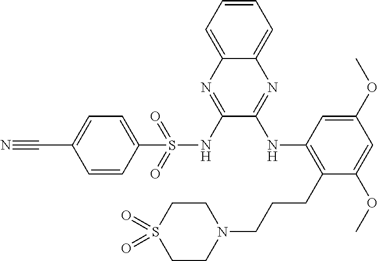 [SMILES: COc1cc(Nc2nc3ccccc3nc2NS(=O)(=O)c2ccc(C#N)cc2)c(CCCN2CCS(=O)(=O)CC2)c(OC)c1] |
| I-6 | 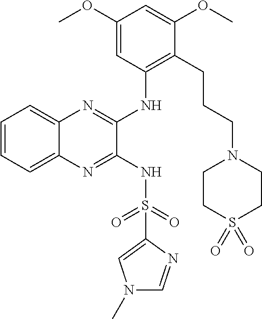 [SMILES: COc1cc(Nc2nc3ccccc3nc2NS(=O)(=O)c2cn(C)cn2)c(CCCN2CCS(=O)(=O)CC2)c(OC)c1] |
| I-7 | 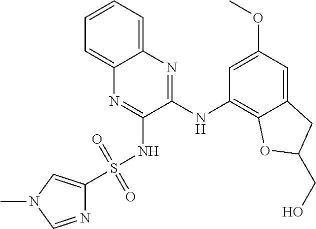 [SMILES: COc1cc2c(c(Nc3nc4ccccc4nc3NS(=O)(=O)c3cn(C)cn3)c1)OC(CO)C2] |
| I-8 | 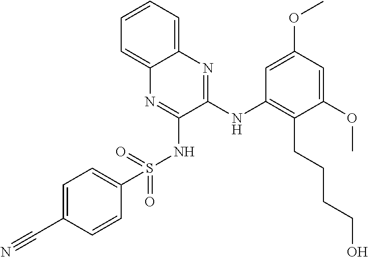 [SMILES: COc1cc(Nc2nc3ccccc3nc2NS(=O)(=O)c2ccc(C#N)cc2)c(CCCCO)c(OC)c1] |
| I-9 | 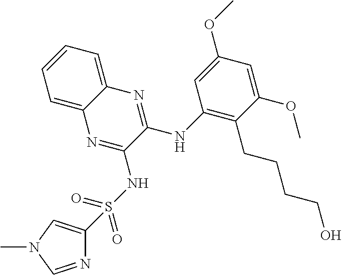 [SMILES: COc1cc(Nc2nc3ccccc3nc2NS(=O)(=O)c2cn(C)cn2)c(CCCCO)c(OC)c1] |
| I-10 | 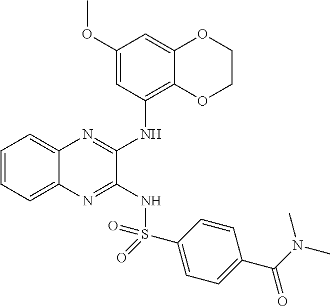 [SMILES: COc1cc(Nc2nc3ccccc3nc2NS(=O)(=O)c2ccc(C(=O)N(C)C)cc2)c2c(c1)OCCO2] |
| I-11 | 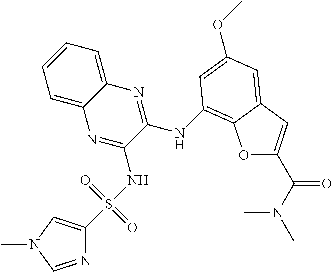 [SMILES: COc1cc(Nc2nc3ccccc3nc2NS(=O)(=O)c2cn(C)cn2)c2oc(C(=O)N(C)C)cc2c1] |
| I-12 | 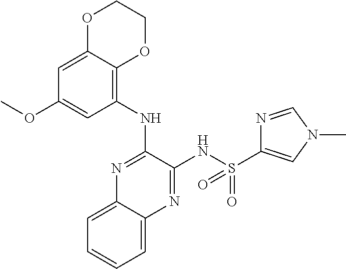 |
| I-13 | 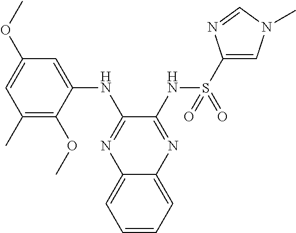 [SMILES: COc1cc(C)c(OC)c(Nc2nc3ccccc3nc2NS(=O)(=O)c2cn(C)cn2)c1] |
| I-14 | 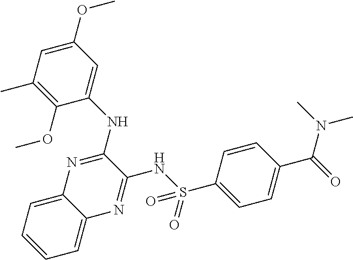 |
| I-15 | 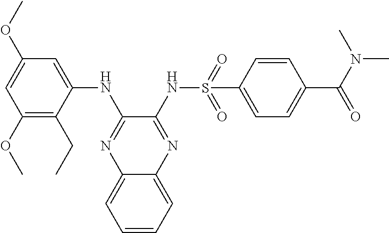 [SMILES: CCc1c(Nc2nc3ccccc3nc2NS(=O)(=O)c2ccc(C(=O)N(C)C)cc2)cc(OC)cc1OC] |
| I-16 | 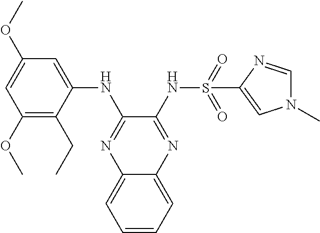 [SMILES: CCc1c(Nc2nc3ccccc3nc2NS(=O)(=O)c2cn(C)cn2)cc(OC)cc1OC] |
| I-17 | 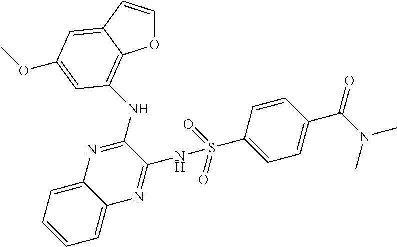 |
| I-18 | 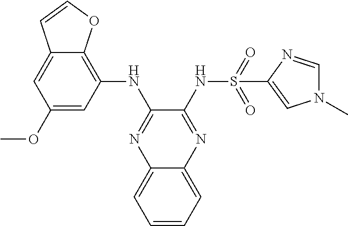 |
| I-19 | 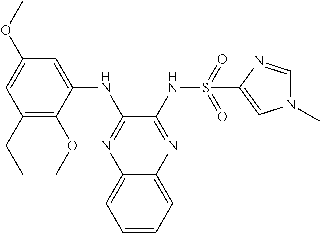 [SMILES: CCc1cc(OC)cc(Nc2nc3ccccc3nc2NS(=O)(=O)c2cn(C)cn2)c1OC] |
| I-20 | 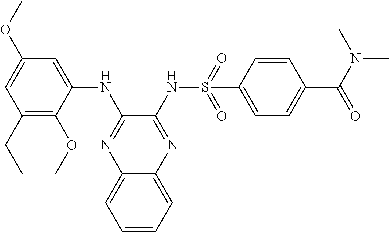 [SMILES: CCc1cc(OC)cc(Nc2nc3ccccc3nc2NS(=O)(=O)c2ccc(C(=O)N(C)C)cc2)c1OC] |
| I-21 | 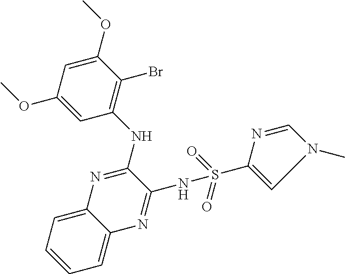 |
| I-22 | 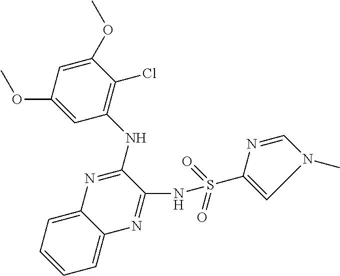 |
| I-23 | 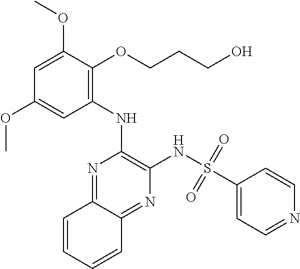 [SMILES: COc1cc(Nc2nc3ccccc3nc2NS(=O)(=O)c2ccncc2)c(OCCCO)c(OC)c1] |
| I-24 | 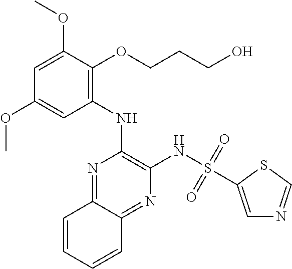 [SMILES: COc1cc(Nc2nc3ccccc3nc2NS(=O)(=O)c2cncs2)c(OCCCO)c(OC)c1] |
| I-25 | 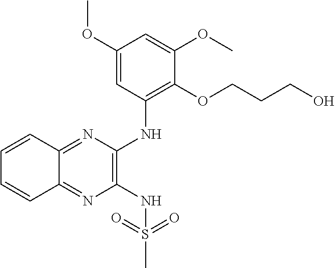 [SMILES: COc1cc(Nc2nc3ccccc3nc2NS(C)(=O)=O)c(OCCCO)c(OC)c1] |
| I-27 | 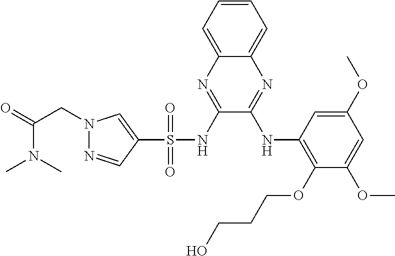 [SMILES: COc1cc(Nc2nc3ccccc3nc2NS(=O)(=O)c2cnn(CC(=O)N(C)C)c2)c(OCCCO)c(OC)c1] |
| I-28 | 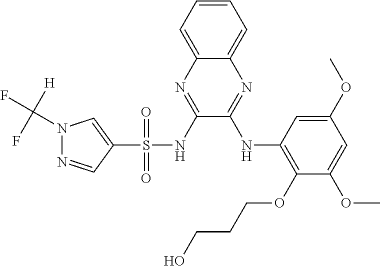 [SMILES: COc1cc(Nc2nc3ccccc3nc2NS(=O)(=O)c2cnn(C(F)F)c2)c(OCCCO)c(OC)c1] |
| I-29 | 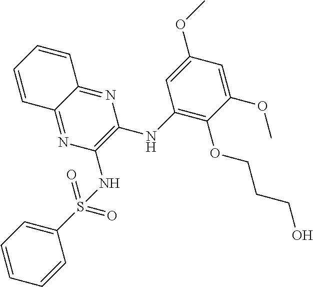 [SMILES: COc1cc(Nc2nc3ccccc3nc2NS(=O)(=O)c2ccccc2)c(OCCCO)c(OC)c1] |
| I-30 | 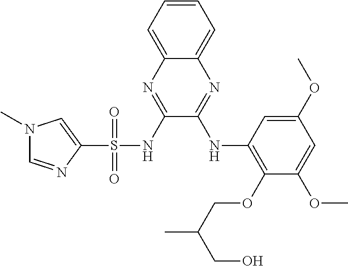 |
| I-31 | 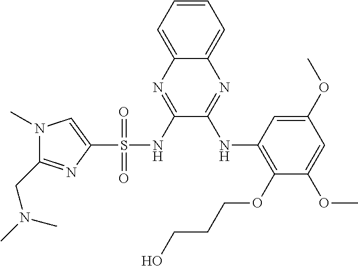 |
| I-32 | 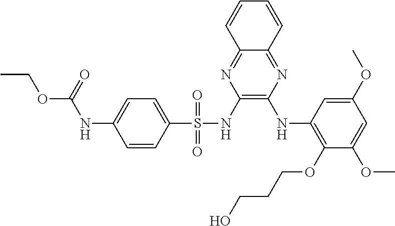 [SMILES: CCOC(=O)Nc1ccc(S(=O)(=O)Nc2nc3ccccc3nc2Nc2cc(OC)cc(OC)c2OCCCO)cc1] |
| I-33 | 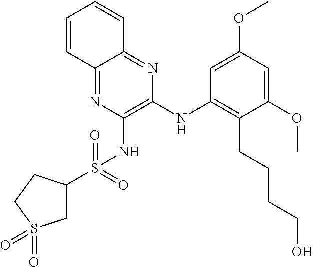 [SMILES: COc1cc(Nc2nc3ccccc3nc2NS(=O)(=O)C2CCS(=O)(=O)C2)c(CCCCO)c(OC)c1] |
| I-34 | 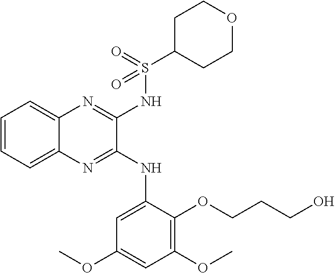 [SMILES: COc1cc(Nc2nc3ccccc3nc2NS(=O)(=O)C2CCOCC2)c(OCCCO)c(OC)c1] |
| I-35 | 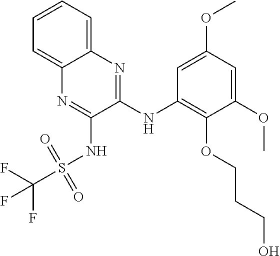 [SMILES: COc1cc(Nc2nc3ccccc3nc2NS(=O)(=O)C(F)(F)F)c(OCCCO)c(OC)c1] |
| I-36 | 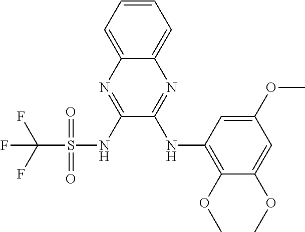 [SMILES: COc1cc(Nc2nc3ccccc3nc2NS(=O)(=O)C(F)(F)F)c(OC)c(OC)c1] |
| I-37 | 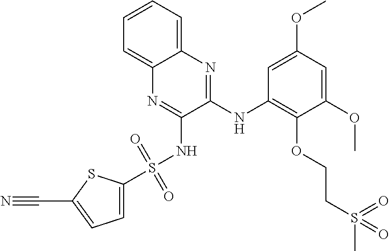 [SMILES: COc1cc(Nc2nc3ccccc3nc2NS(=O)(=O)c2ccc(C#N)s2)c(OCCS(C)(=O)=O)c(OC)c1] |
| I-38 | 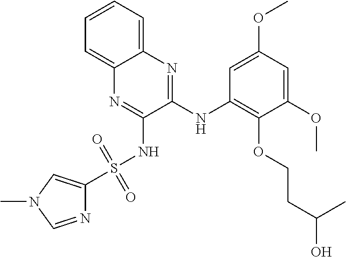 [SMILES: COc1cc(Nc2nc3ccccc3nc2NS(=O)(=O)c2cn(C)cn2)c(OCCC(C)O)c(OC)c1] |
| I-39 | 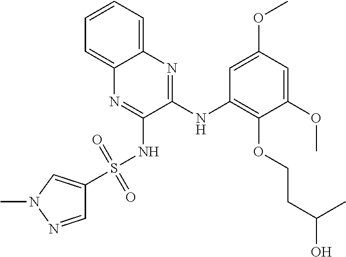 [SMILES: COc1cc(Nc2nc3ccccc3nc2NS(=O)(=O)c2cnn(C)c2)c(OCCC(C)O)c(OC)c1] |
| I-40 | 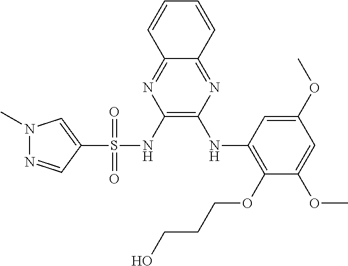 |
| I-41 | 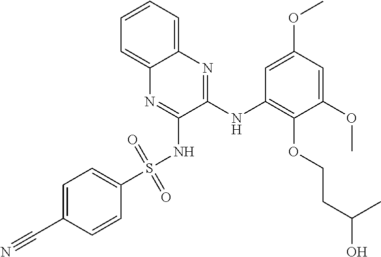 [SMILES: COc1cc(Nc2nc3ccccc3nc2NS(=O)(=O)c2ccc(C#N)cc2)c(OCCC(C)O)c(OC)c1] |
| I-42 | 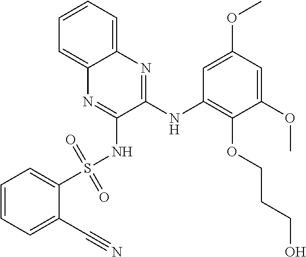 [SMILES: COc1cc(Nc2nc3ccccc3nc2NS(=O)(=O)c2ccccc2C#N)c(OCCCO)c(OC)c1] |
| I-43 | 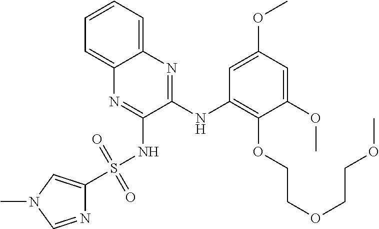 |
| I-44 | 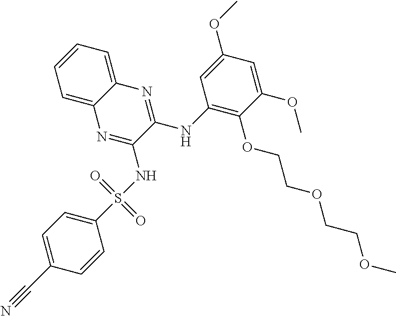 [SMILES: COCCOCCOc1c(Nc2nc3ccccc3nc2NS(=O)(=O)c2ccc(C#N)cc2)cc(OC)cc1OC] |
| I-45 | 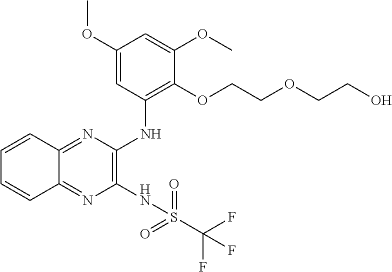 [SMILES: COc1cc(Nc2nc3ccccc3nc2NS(=O)(=O)C(F)(F)F)c(OCCOCCO)c(OC)c1] |
| I-46 | 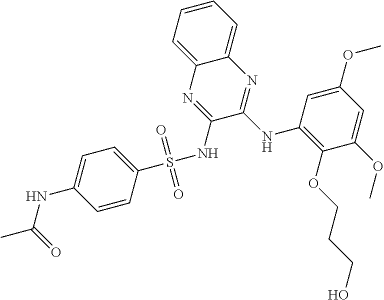 [SMILES: COc1cc(Nc2nc3ccccc3nc2NS(=O)(=O)c2ccc(NC(C)=O)cc2)c(OCCCO)c(OC)c1] |
| I-47 | 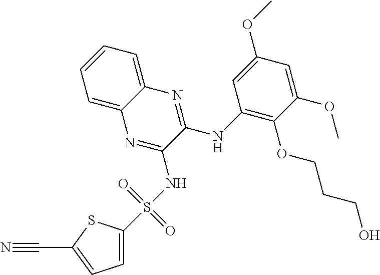 [SMILES: COc1cc(Nc2nc3ccccc3nc2NS(=O)(=O)c2ccc(C#N)s2)c(OCCCO)c(OC)c1] |
| I-48 | 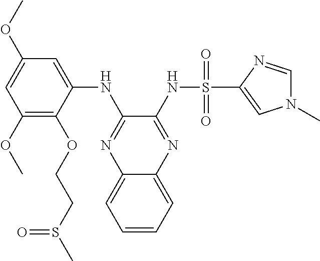 [SMILES: COc1cc(Nc2nc3ccccc3nc2NS(=O)(=O)c2cn(C)cn2)c(OCCS(C)=O)c(OC)c1] |
| I-49 | 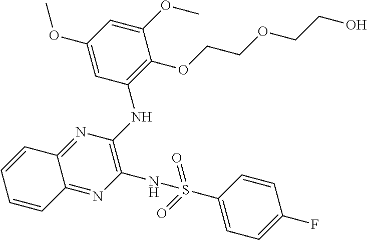 [SMILES: COc1cc(Nc2nc3ccccc3nc2NS(=O)(=O)c2ccc(F)cc2)c(OCCOCCO)c(OC)c1] |
| I-50 | 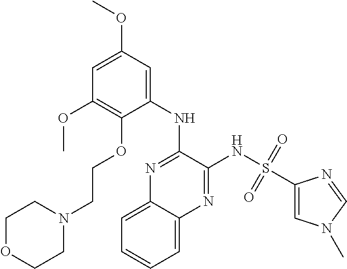 [SMILES: COc1cc(Nc2nc3ccccc3nc2NS(=O)(=O)c2cn(C)cn2)c(OCCN2CCOCC2)c(OC)c1] |
| I-51 | 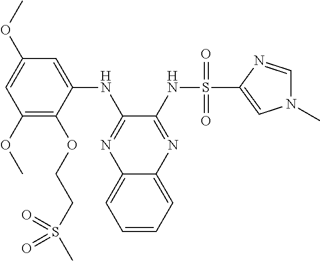 [SMILES: COc1cc(Nc2nc3ccccc3nc2NS(=O)(=O)c2cn(C)cn2)c(OCCS(C)(=O)=O)c(OC)c1] |
| I-52 | 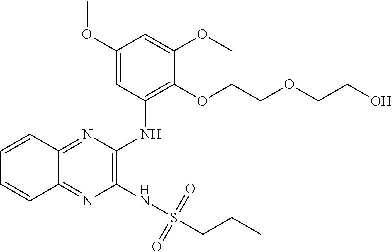 [SMILES: CCCS(=O)(=O)Nc1nc2ccccc2nc1Nc1cc(OC)cc(OC)c1OCCOCCO] |
| I-53 | 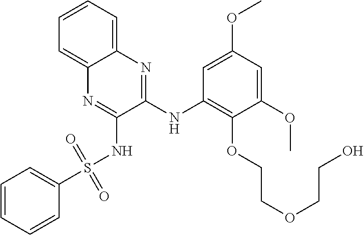 [SMILES: COc1cc(Nc2nc3ccccc3nc2NS(=O)(=O)c2ccccc2)c(OCCOCCO)c(OC)c1] |
| I-54 | 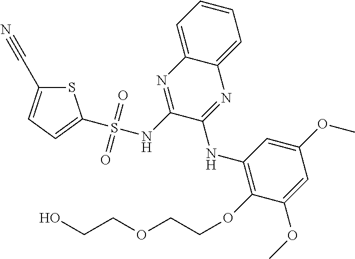 [SMILES: COc1cc(Nc2nc3ccccc3nc2NS(=O)(=O)c2ccc(C#N)s2)c(OCCOCCO)c(OC)c1] |
| I-55 | 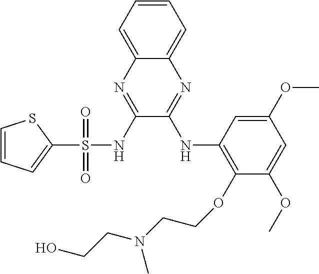 [SMILES: COc1cc(Nc2nc3ccccc3nc2NS(=O)(=O)c2cccs2)c(OCCN(C)CCO)c(OC)c1] |
| I-56 | 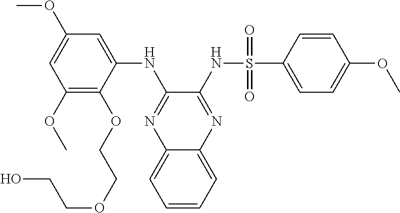 |
| I-57 | 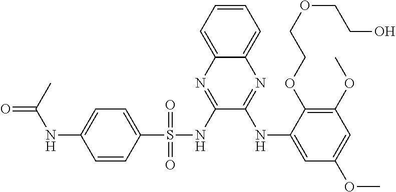 [SMILES: COc1cc(Nc2nc3ccccc3nc2NS(=O)(=O)c2ccc(NC(C)=O)cc2)c(OCCOCCO)c(OC)c1] |
| I-58 | 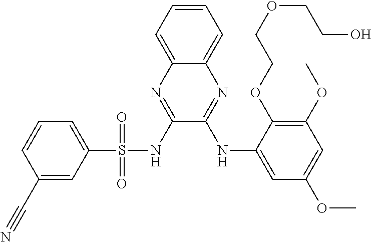 [SMILES: COc1cc(Nc2nc3ccccc3nc2NS(=O)(=O)c2cccc(C#N)c2)c(OCCOCCO)c(OC)c1] |
| I-59 | 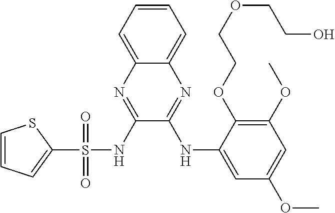 [SMILES: COc1cc(Nc2nc3ccccc3nc2NS(=O)(=O)c2cccs2)c(OCCOCCO)c(OC)c1] |
| I-60 | 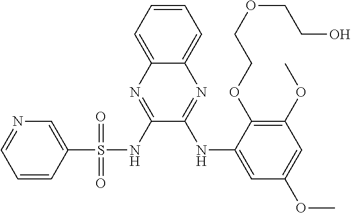 [SMILES: COc1cc(Nc2nc3ccccc3nc2NS(=O)(=O)c2cccnc2)c(OCCOCCO)c(OC)c1] |
| I-61 | 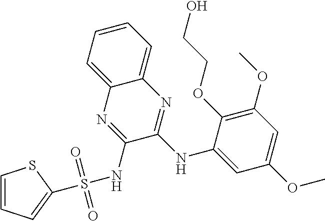 [SMILES: COc1cc(Nc2nc3ccccc3nc2NS(=O)(=O)c2cccs2)c(OCCO)c(OC)c1] |
| I-62 | 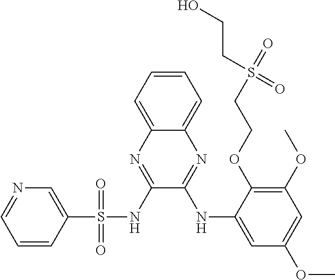 [SMILES: COc1cc(Nc2nc3ccccc3nc2NS(=O)(=O)c2cccnc2)c(OCCS(=O)(=O)CCO)c(OC)c1] |
| I-63 | 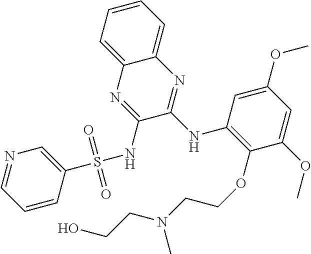 [SMILES: COc1cc(Nc2nc3ccccc3nc2NS(=O)(=O)c2cccnc2)c(OCCN(C)CCO)c(OC)c1] |
| I-64 | 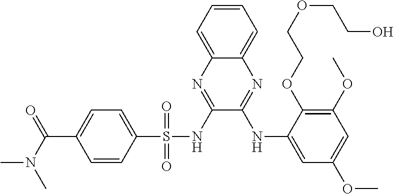 [SMILES: COc1cc(Nc2nc3ccccc3nc2NS(=O)(=O)c2ccc(C(=O)N(C)C)cc2)c(OCCOCCO)c(OC)c1] |
| I-65 | 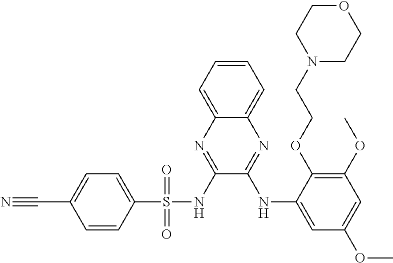 [SMILES: COc1cc(Nc2nc3ccccc3nc2NS(=O)(=O)c2ccc(C#N)cc2)c(OCCN2CCOCC2)c(OC)c1] |
| I-66 | 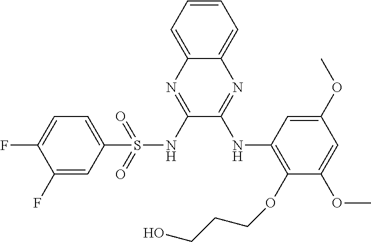 [SMILES: COc1cc(Nc2nc3ccccc3nc2NS(=O)(=O)c2ccc(F)c(F)c2)c(OCCCO)c(OC)c1] |
| I-67 | 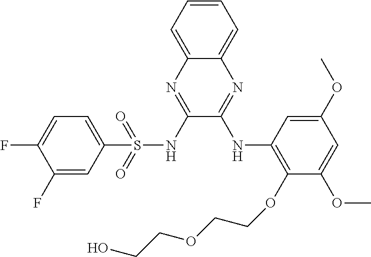 [SMILES: COc1cc(Nc2nc3ccccc3nc2NS(=O)(=O)c2ccc(F)c(F)c2)c(OCCOCCO)c(OC)c1] |
| I-68 | 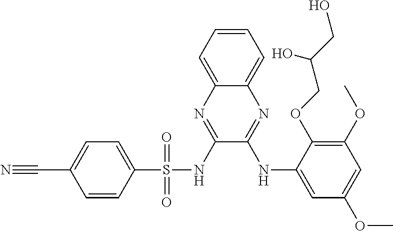 [SMILES: COc1cc(Nc2nc3ccccc3nc2NS(=O)(=O)c2ccc(C#N)cc2)c(OCC(O)CO)c(OC)c1] |
| I-69 | 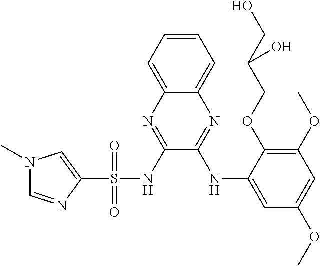 [SMILES: COc1cc(Nc2nc3ccccc3nc2NS(=O)(=O)c2cn(C)cn2)c(OCC(O)CO)c(OC)c1] |
| I-70 | 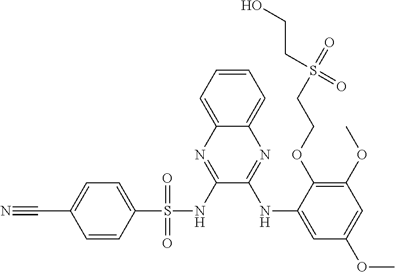 [SMILES: COc1cc(Nc2nc3ccccc3nc2NS(=O)(=O)c2ccc(C#N)cc2)c(OCCS(=O)(=O)CCO)c(OC)c1] |
| I-71 | 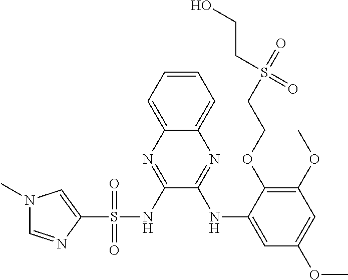 |
| I-72 | 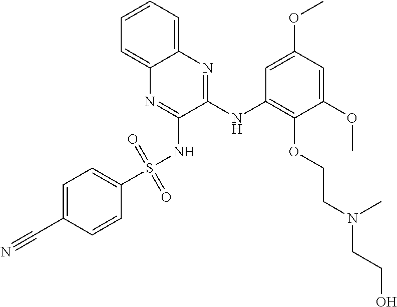 [SMILES: COc1cc(Nc2nc3ccccc3nc2NS(=O)(=O)c2ccc(C#N)cc2)c(OCCN(C)CCO)c(OC)c1] |
| I-73 | 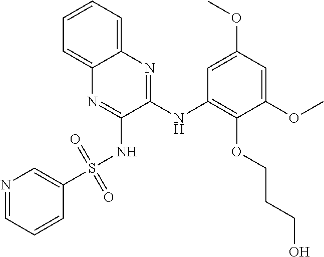 [SMILES: COc1cc(Nc2nc3ccccc3nc2NS(=O)(=O)c2cccnc2)c(OCCCO)c(OC)c1] |
| I-74 | 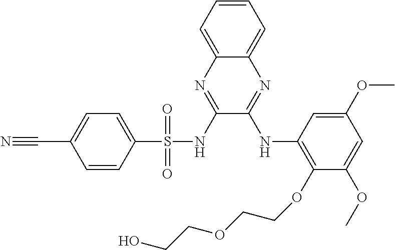 [SMILES: COc1cc(Nc2nc3ccccc3nc2NS(=O)(=O)c2ccc(C#N)cc2)c(OCCOCCO)c(OC)c1] |
| I-75 | 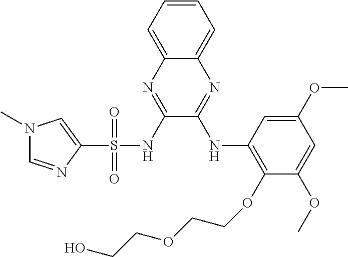 |
| I-76 | 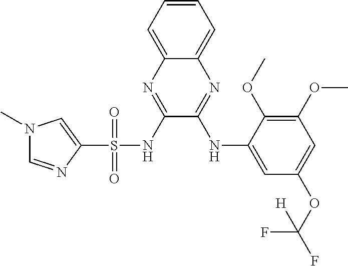 |
| I-77 | 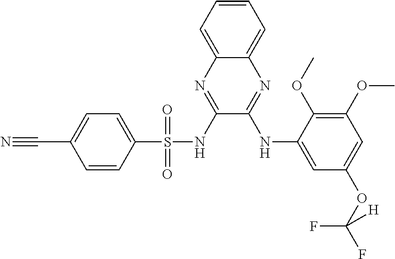 [SMILES: COc1cc(OC(F)F)cc(Nc2nc3ccccc3nc2NS(=O)(=O)c2ccc(C#N)cc2)c1OC] |
| I-78 | 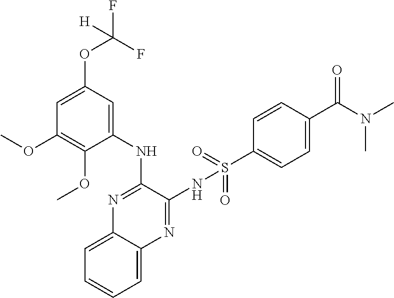 |
| I-79 | 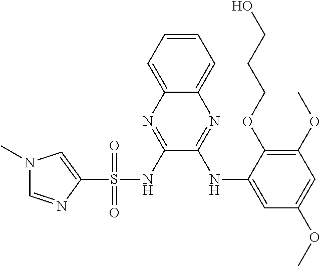 [SMILES: COc1cc(Nc2nc3ccccc3nc2NS(=O)(=O)c2cn(C)cn2)c(OCCCO)c(OC)c1] |
| I-80 | 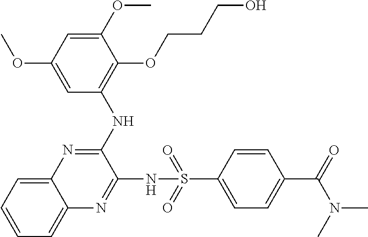 [SMILES: COc1cc(Nc2nc3ccccc3nc2NS(=O)(=O)c2ccc(C(=O)N(C)C)cc2)c(OCCCO)c(OC)c1] |
| I-81 | 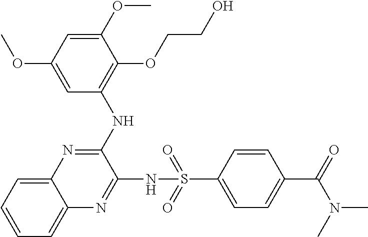 [SMILES: COc1cc(Nc2nc3ccccc3nc2NS(=O)(=O)c2ccc(C(=O)N(C)C)cc2)c(OCCO)c(OC)c1] |
| I-82 | 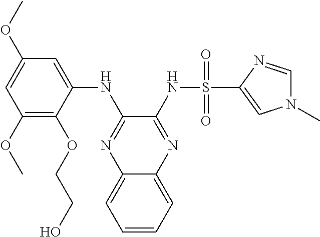 [SMILES: COc1cc(Nc2nc3ccccc3nc2NS(=O)(=O)c2cn(C)cn2)c(OCCO)c(OC)c1] |
| I-83 | 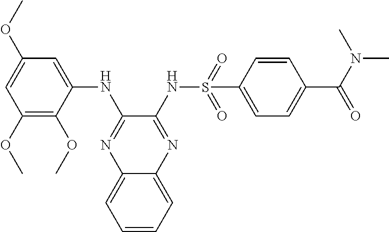 [SMILES: COc1cc(Nc2nc3ccccc3nc2NS(=O)(=O)c2ccc(C(=O)N(C)C)cc2)c(OC)c(OC)c1] |
| I-84 | 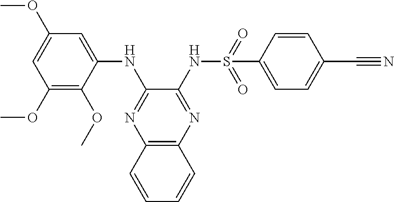 [SMILES: COc1cc(Nc2nc3ccccc3nc2NS(=O)(=O)c2ccc(C#N)cc2)c(OC)c(OC)c1] |
| I-85 | 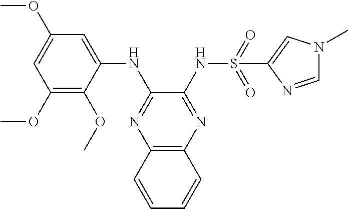 |
| I-86 | 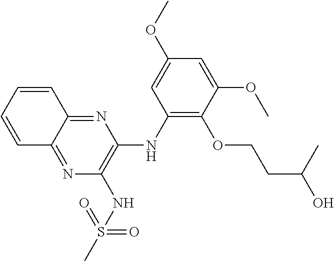 [SMILES: COc1cc(Nc2nc3ccccc3nc2NS(C)(=O)=O)c(OCCC(C)O)c(OC)c1] |

Reference 1: Preparation of quinoxaline derivatives for use as therapeutic agents for autoimmune disorders. Gaillard, Pascale; Pomel, Vincent; Jeanclaude-Etter, Isabelle; Dorbais, Jerome; Klicic, Jasna; Montagne, Cyril. PCT Int. Appl. (2008), 211 pp. WO 2008101979 A1
Reference 2: Preparation of pyrazine derivatives, particularly N-[3-(oxyphenylamino)quinoxalin-2-yl]sulfonamides, as PI3K inhibitors. Gaillard, Pascale; Quattropani, Anna; Pomel, Vincent; Rueckle, Thomas; Klicic, Jasna; Church, Dennis. (Applied Research Systems Ars Holding N.V., Neth. Antilles). PCT Int. Appl. (2007), 170 pp. WO 2007023186 A1
Reference 3: Preparation of substituted N-(pyrazin-2-yl)benzenesulfonamides and related compounds as CRTH2 modulators, particularly inhibitors, and their use for treating allergic and immune diseases, inflammatory dermatosis and neurodegenerative disorders. Page, Patrick; Schwarz, Matthias; Sebille, Eric; Cleva, Christophe; Merlot, Cedric; Maio, Maurizio. (Applied Research Systems ARS Holding N.V., Neth. Antilles). PCT Int. Appl. (2006), 112 pp. WO 2006111560 A2
Reference 4: Preparation of condensed n-pyrazinyl-sulfonamides and their use in the treatment of chemokine mediated diseases. Baxter, Andrew; Kindon, Nicholas; Stocks, Michael. (Astrazeneca Ab, Swed.). PCT Int. Appl. (2005), 48 pp. WO 2005021513 A1
Reference 5: Preparation of 2,3-substituted pyrazine derivatives capable of binding to G-protein coupled receptors. Jones, Graham Peter; Hardy, David; Macritchie, Jacqueline Anne; Slater, Martin John. (Biofocus Plc, UK). PCT Int. Appl. (2004), 102 pp. WO 2004058265
Reference 6: Preparation of N-pyrazinylthiophenesulfonamides as chemokine receptor modulators. Baxter, Andrew; Johnson, Timothy; Kindon, Nicholas; Roberts, Bryan; Steele, John; Stocks, Michael; Tomkinson, Nicholas. (Astrazeneca A B, Swed.). PCT Int. Appl. (2003), 51 pp. WO 2003051870 A1
Reference 7: Synthesis, Structure, and chemical properties of some N-(3-chloro-2-quinoxalyl)arylsulfonamides. S. V. Litvinenko, V. I. Savich, mid L. D. Bobrovnik Chemistry of Heterocyclic Compounds, Vol. 30, No. 3, 1994
Reference 8: Lamb, Peter; Matthews, David. Quinaxoline derivatives as inhibitors of PI3K-alpha and their preparation, pharmaceutical compositions and use in the treatment of cancer. PCT Int. Appl. (2008), 496 pp. WO 2008127594 A2
Reference 9: Bajjalieh, William; Bannen, Lynne Canne; Brown, S. David; Kearney, Patrick; Mac, Morrison; Marlowe, Charles K.; Nuss, John M.; Tesfai, Zerom; Wang, Yong; Xu, Wei. 2-Amino-3-sulfonylaminoquinoxaline derivatives as phosphatidylinositol 3-kinase inhibitors and their preparation, pharmaceutical compositions and use in the treatment of cancer. PCT Int. Appl. (2007), 296 pp. WO 2007044729 A2

1. A process for the preparation of compounds of formulae (I) or (I′): 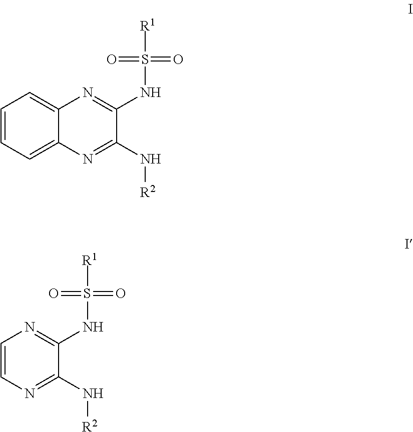
wherein
R¹ is selected from the group consisting of A, C₃-C₈-cylcoalkyl, Het, and Ar.
R² is selected from the group consisting of Ar and Het.
Ar denotes a monocyclic or bicyclic, aromatic carbocyclic ring having 6 to 14 carbon atoms, which is unsubstituted or monosubstituted, disubstituted or trisubstituted by Hal, CF₃, OCF₃, NO₂, CN, perfluoroalkyl, A, —OR⁶, —NHR⁶, —COR^{E}, —CONHR⁶, —CON(R⁶)₂, —NR⁶COR⁶, —NR⁶CO₂R⁶, —NR⁶SO₂A, NR⁶CONR′R″, —COOR⁶, —SO₂A, —SO₂NR⁶A, —SO₂Het, —SO₂NR⁶Het, Ar, Het, —NR⁶SO₂NR⁶Het, COHet, COAr, or C₃-C₈-cycloalkyl.
Het denotes a monocyclic or bicyclic saturated, unsaturated or aromatic heterocyclic ring having 1 to 4 N, O and/or S atoms and/or 1 group selected from CO, SO or SO₂, which is unsubstituted or monosubstituted, disubstituted or trisubstituted by Hal, CF₃, OCF₃, NO₂, CN, perfluoroalkyl, A, —OR⁶, —NHR⁶, —COR^{E}, —CONHR⁶, —CON(R⁶)₂, —NR⁶COR⁶, —NR⁶CO₂R⁶, —NR⁶SO₂A, NR⁶CONR′R″, —COOR⁶, —SO₂A, —SO₂NR⁶A, —SO₂Het, —SO₂NR⁶Het, Ar, Het, —NR⁶SO₂NR⁶Het, or C₃-C₈-cycloalkyl.
A is a branched or linear alkyl having 1 to 12 C-atoms, wherein one or more, preferably 1 to 7H-atoms may be replaced by Hal, Ar, Het, OR⁶, CN, NR⁶COA, CONR′R″, COOR⁶ or NR′R″ and wherein one or more, preferably 1 to 7 non-adjacent CH₂-groups may be replaced by O, NR⁶ or S and/or by —CH═CH— or —C≡C— groups, or denotes cycloalkyl, cycloalken or cycloalkylalkylen having 3-7 ring C atoms wherein the cycloalkylen is optionally substituted by 1 to 3 groups selected from OR⁶, Hal, Ar, Het, CN, NR⁶COA, CONR′R″, COOR⁶;
R′, R″ denote independently from each other H, A, Ar, or Het,
R⁶ is H or A.
comprising
step a) the reaction of 2,3-dichloroquinoxaline with a compound of formula (III) in a polar aprotic solvent, in the presence of an alkyli metal hydroxide, 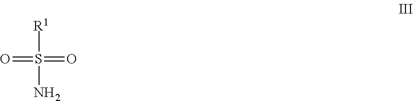
to provide a compound of Formula (II) or (II′): 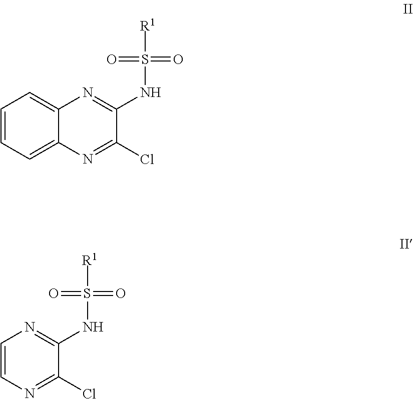
and,
step b) the reaction of compounds of Formula (II) with a amine of formula NH₂R².

2. The process as defined in claim 1 wherein the polar aprotic solvent is selected from DMA, DMF, NMP and DMSO.

3. The process of claim 1 wherein the alkali metal hydroxide is selected from LiOH and KOH.

4. The process of claim 1 wherein the step b) is performed in the presence of a pyridine base.

5. The process of claim 4 wherein the pyridine base is selected from pyridine, methyl pyridine, and 2,6-di-methylpyridine.

6. The process of claim 4 wherein the pyridine base is lutidine.

7. The process of claim 1 wherein step b) is performed in a polar solvent.

8. The process of claim 7 wherein the polar solvent is selected from DMA, DMF, NMP, DMSO or alcohol.

9. The process of claim 1 wherein the compound of Formula (II) is selected from the following group:

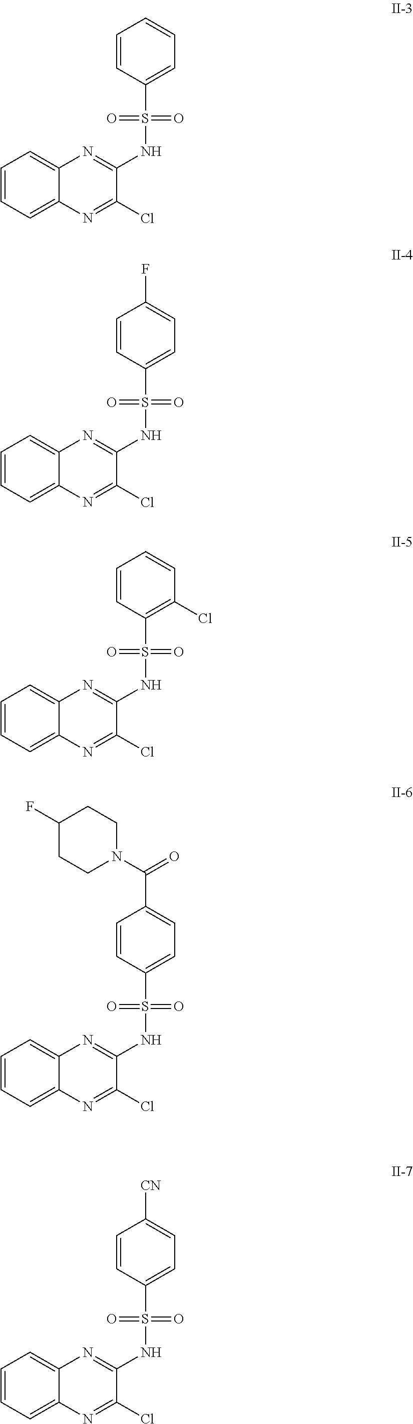 [SMILES: O=S(=O)(Nc1nc2ccccc2nc1Cl)c1ccccc1 ; O=S(=O)(Nc1nc2ccccc2nc1Cl)c1ccc(F)cc1 ; O=S(=O)(Nc1nc2ccccc2nc1Cl)c1ccccc1Cl ; O=C(c1ccc(S(=O)(=O)Nc2nc3ccccc3nc2Cl)cc1)N1CCC(F)CC1 ; N#Cc1ccc(S(=O)(=O)Nc2nc3ccccc3nc2Cl)cc1]

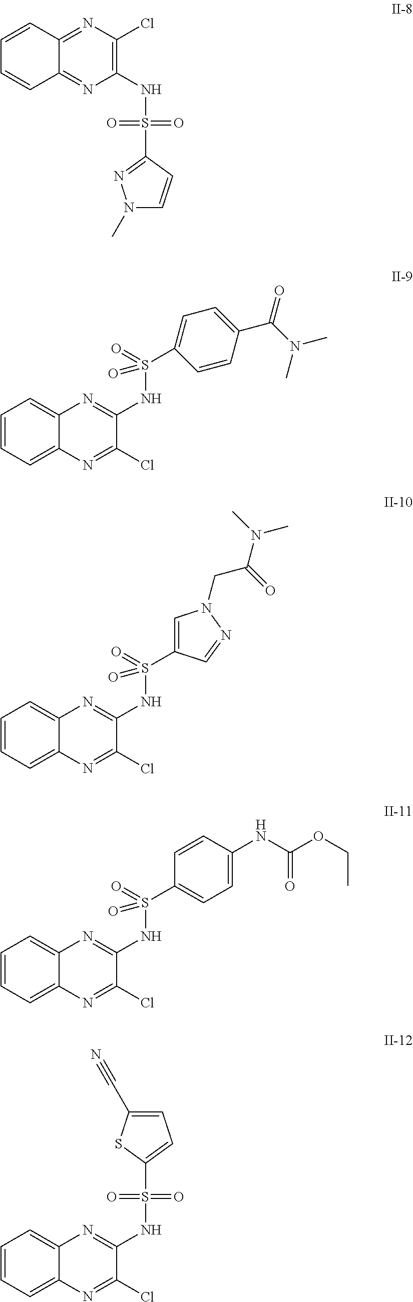 [SMILES: Cn1ccc(S(=O)(=O)Nc2nc3ccccc3nc2Cl)n1 ; CN(C)C(=O)c1ccc(S(=O)(=O)Nc2nc3ccccc3nc2Cl)cc1 ; CN(C)C(=O)Cn1cc(S(=O)(=O)Nc2nc3ccccc3nc2Cl)cn1 ; CCOC(=O)Nc1ccc(S(=O)(=O)Nc2nc3ccccc3nc2Cl)cc1 ; N#Cc1ccc(S(=O)(=O)Nc2nc3ccccc3nc2Cl)s1]

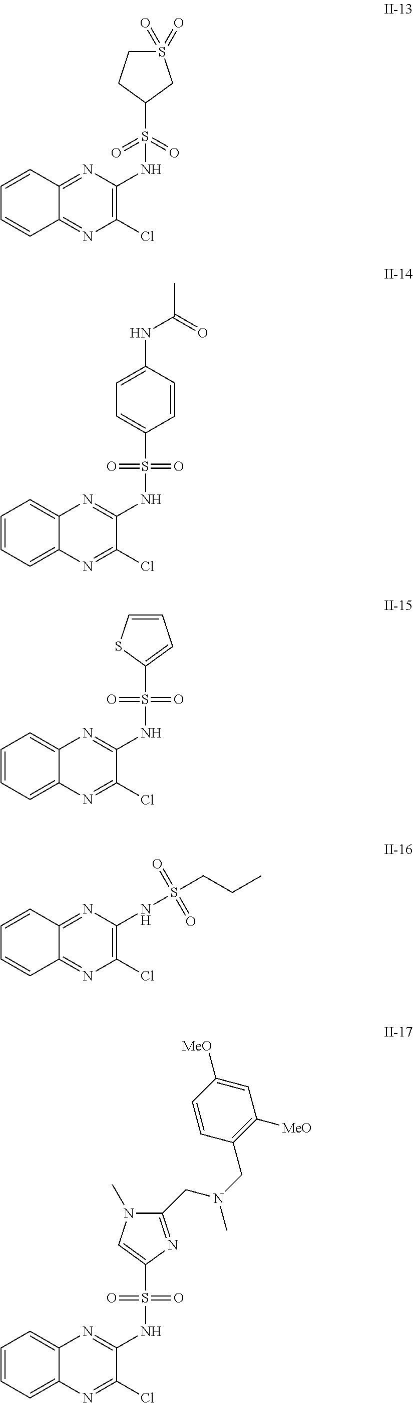 [SMILES: O=S1(=O)CCC(S(=O)(=O)Nc2nc3ccccc3nc2Cl)C1 ; CC(=O)Nc1ccc(S(=O)(=O)Nc2nc3ccccc3nc2Cl)cc1 ; O=S(=O)(Nc1nc2ccccc2nc1Cl)c1cccs1 ; CCCS(=O)(=O)Nc1nc2ccccc2nc1Cl ; COc1ccc(CN(C)Cc2nc(S(=O)(=O)Nc3nc4ccccc4nc3Cl)cn2C)c(OC)c1]

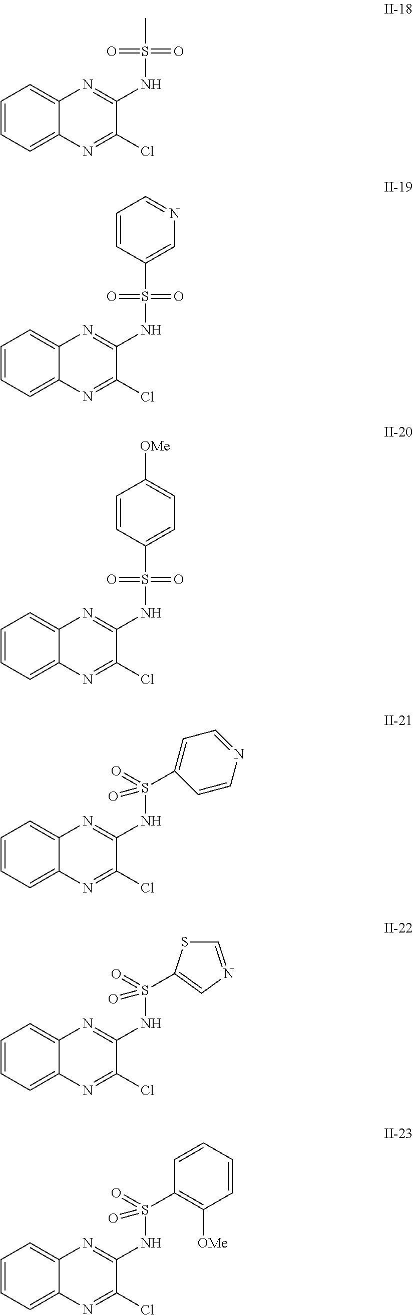

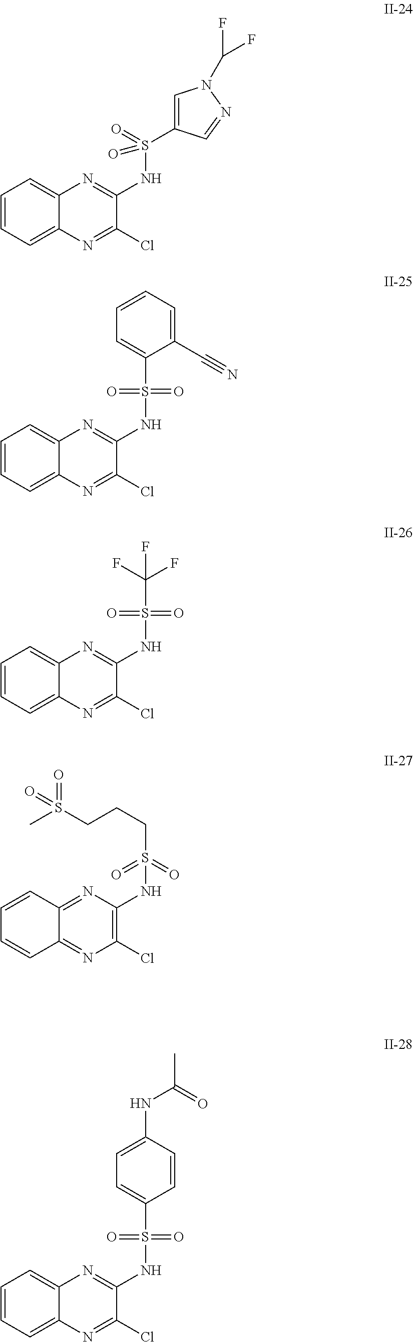

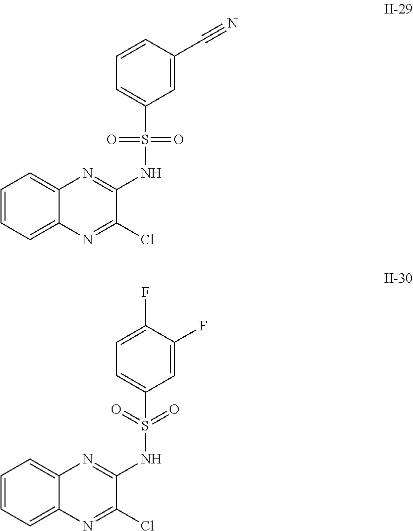 [SMILES: N#Cc1cccc(S(=O)(=O)Nc2nc3ccccc3nc2Cl)c1 ; O=S(=O)(Nc1nc2ccccc2nc1Cl)c1ccc(F)c(F)c1]

10. The process of claim 1 wherein the compound of Formula (II′) is

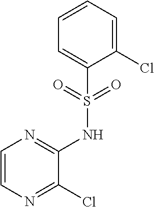 [SMILES: O=S(=O)(Nc1nccnc1Cl)c1ccccc1Cl]

11. The process as defined in claim 1 wherein the compound of Formula (I) is selected from the following group:

| Ex. | Structure |
|---|---|
| I-3 | 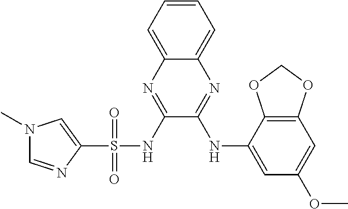 |
| I-4 | 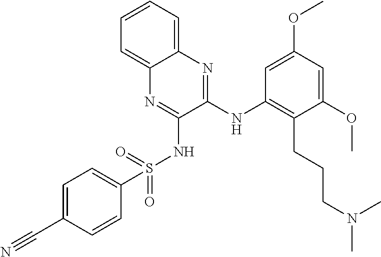 [SMILES: COc1cc(Nc2nc3ccccc3nc2NS(=O)(=O)c2ccc(C#N)cc2)c(CCCN(C)C)c(OC)c1] |
| I-5 | 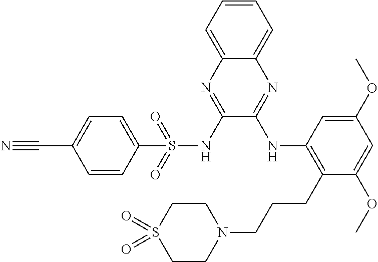 [SMILES: COc1cc(Nc2nc3ccccc3nc2NS(=O)(=O)c2ccc(C#N)cc2)c(CCCN2CCS(=O)(=O)CC2)c(OC)c1] |
| I-6 | 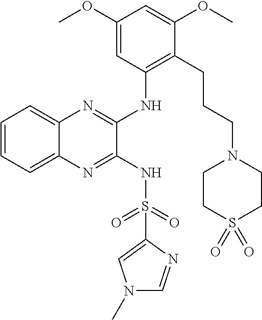 [SMILES: COc1cc(Nc2nc3ccccc3nc2NS(=O)(=O)c2cn(C)cn2)c(CCCN2CCS(=O)(=O)CC2)c(OC)c1] |
| I-7 | 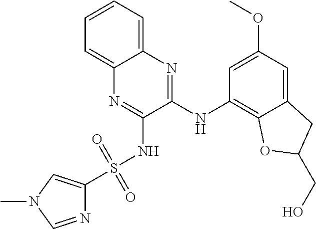 [SMILES: COc1cc2c(c(Nc3nc4ccccc4nc3NS(=O)(=O)c3cn(C)cn3)c1)OC(CO)C2] |
| I-8 | 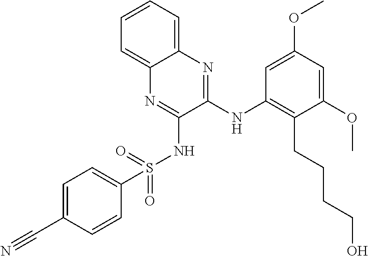 [SMILES: COc1cc(Nc2nc3ccccc3nc2NS(=O)(=O)c2ccc(C#N)cc2)c(CCCCO)c(OC)c1] |
| I-9 | 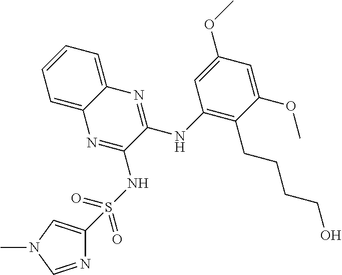 [SMILES: COc1cc(Nc2nc3ccccc3nc2NS(=O)(=O)c2cn(C)cn2)c(CCCCO)c(OC)c1] |
| I-10 | 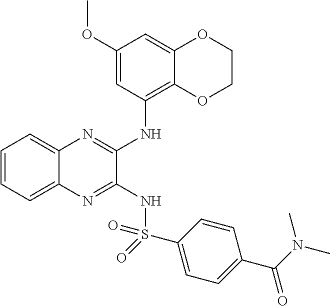 [SMILES: COc1cc(Nc2nc3ccccc3nc2NS(=O)(=O)c2ccc(C(=O)N(C)C)cc2)c2c(c1)OCCO2] |
| I-11 | 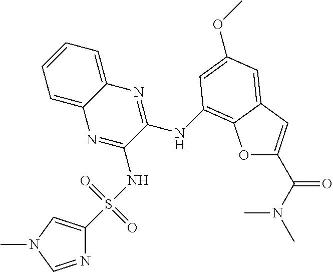 [SMILES: COc1cc(Nc2nc3ccccc3nc2NS(=O)(=O)c2cn(C)cn2)c2oc(C(=O)N(C)C)cc2c1] |
| I-12 | 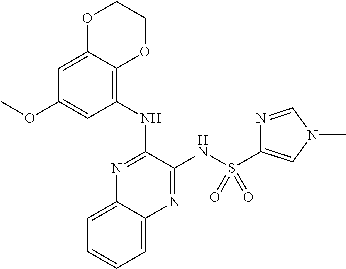 |
| I-13 | 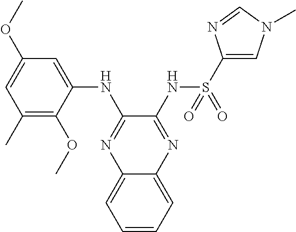 [SMILES: COc1cc(C)c(OC)c(Nc2nc3ccccc3nc2NS(=O)(=O)c2cn(C)cn2)c1] |
| I-14 | 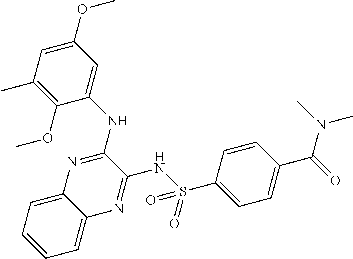 |
| I-15 | 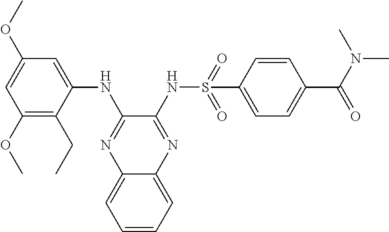 [SMILES: CCc1c(Nc2nc3ccccc3nc2NS(=O)(=O)c2ccc(C(=O)N(C)C)cc2)cc(OC)cc1OC] |
| I-16 | 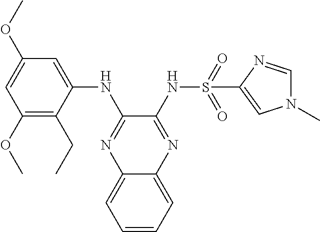 [SMILES: CCc1c(Nc2nc3ccccc3nc2NS(=O)(=O)c2cn(C)cn2)cc(OC)cc1OC] |
| I-17 | 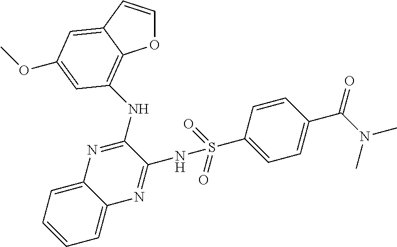 |
| I-18 | 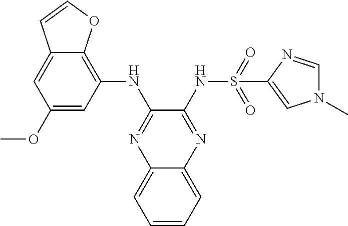 |
| I-19 | 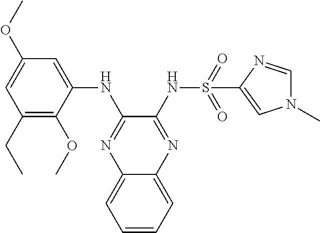 [SMILES: CCc1cc(OC)cc(Nc2nc3ccccc3nc2NS(=O)(=O)c2cn(C)cn2)c1OC] |
| I-20 | 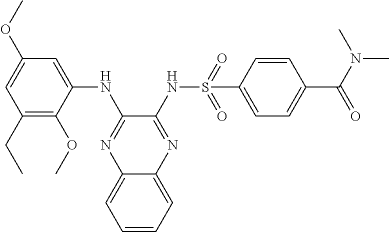 [SMILES: CCc1cc(OC)cc(Nc2nc3ccccc3nc2NS(=O)(=O)c2ccc(C(=O)N(C)C)cc2)c1OC] |
| I-21 | 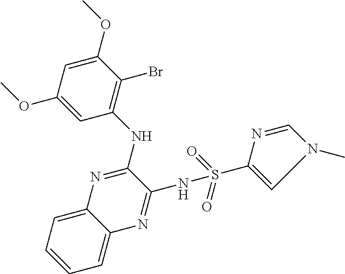 |
| I-22 | 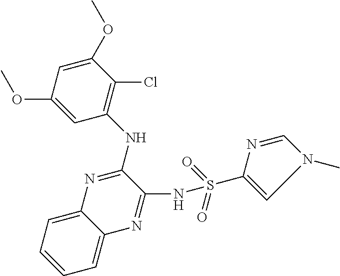 |
| I-23 | 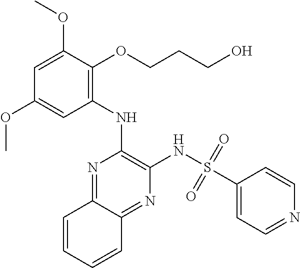 [SMILES: COc1cc(Nc2nc3ccccc3nc2NS(=O)(=O)c2ccncc2)c(OCCCO)c(OC)c1] |
| I-24 | 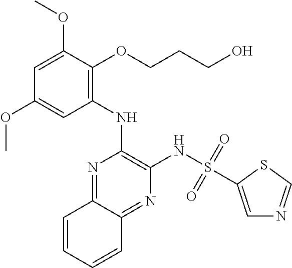 [SMILES: COc1cc(Nc2nc3ccccc3nc2NS(=O)(=O)c2cncs2)c(OCCCO)c(OC)c1] |
| I-25 | 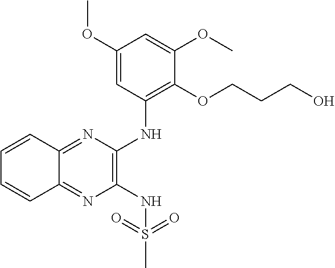 [SMILES: COc1cc(Nc2nc3ccccc3nc2NS(C)(=O)=O)c(OCCCO)c(OC)c1] |
| I-27 | 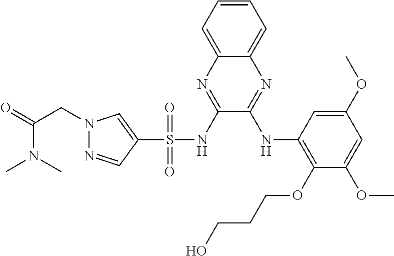 [SMILES: COc1cc(Nc2nc3ccccc3nc2NS(=O)(=O)c2cnn(CC(=O)N(C)C)c2)c(OCCCO)c(OC)c1] |
| I-28 | 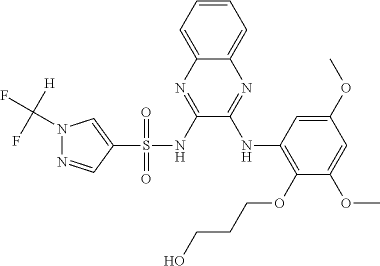 [SMILES: COc1cc(Nc2nc3ccccc3nc2NS(=O)(=O)c2cnn(C(F)F)c2)c(OCCCO)c(OC)c1] |
| I-29 | 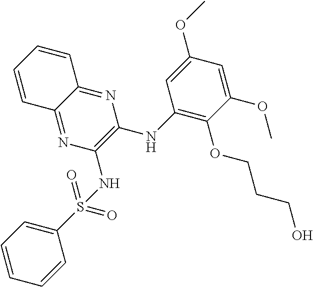 [SMILES: COc1cc(Nc2nc3ccccc3nc2NS(=O)(=O)c2ccccc2)c(OCCCO)c(OC)c1] |
| I-30 | 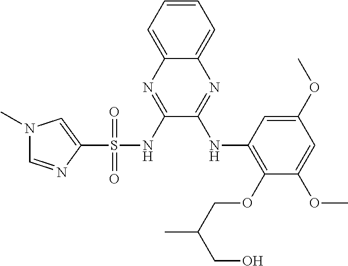 |
| I-31 | 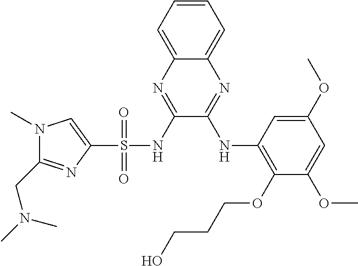 |
| I-32 | 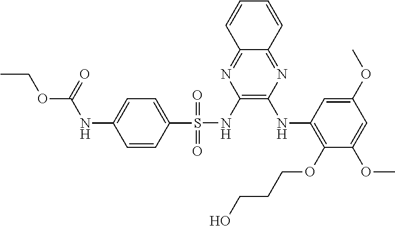 [SMILES: CCOC(=O)Nc1ccc(S(=O)(=O)Nc2nc3ccccc3nc2Nc2cc(OC)cc(OC)c2OCCCO)cc1] |
| I-33 | 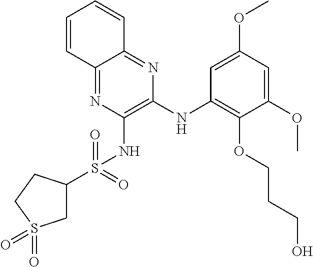 [SMILES: COc1cc(Nc2nc3ccccc3nc2NS(=O)(=O)C2CCS(=O)(=O)C2)c(OCCCO)c(OC)c1] |
| I-34 | 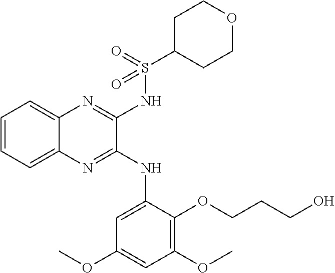 [SMILES: COc1cc(Nc2nc3ccccc3nc2NS(=O)(=O)C2CCOCC2)c(OCCCO)c(OC)c1] |
| I-35 | 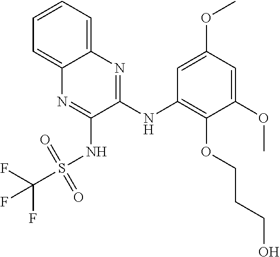 [SMILES: COc1cc(Nc2nc3ccccc3nc2NS(=O)(=O)C(F)(F)F)c(OCCCO)c(OC)c1] |
| I-36 | 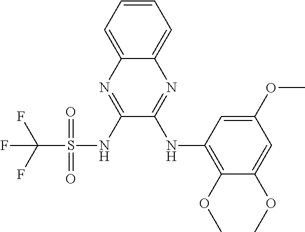 [SMILES: COc1cc(Nc2nc3ccccc3nc2NS(=O)(=O)C(F)(F)F)c(OC)c(OC)c1] |
| I-37 | 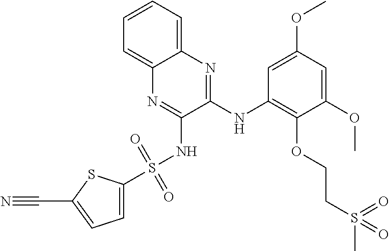 [SMILES: COc1cc(Nc2nc3ccccc3nc2NS(=O)(=O)c2ccc(C#N)s2)c(OCCS(C)(=O)=O)c(OC)c1] |
| I-38 | 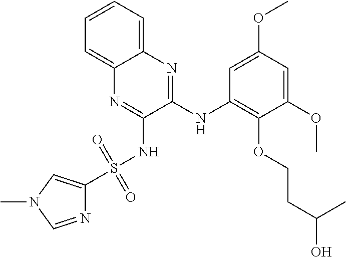 [SMILES: COc1cc(Nc2nc3ccccc3nc2NS(=O)(=O)c2cn(C)cn2)c(OCCC(C)O)c(OC)c1] |
| I-39 | 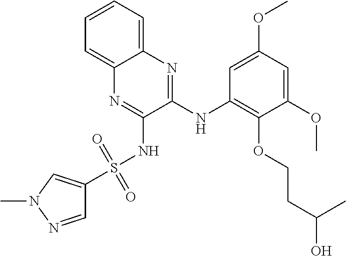 [SMILES: COc1cc(Nc2nc3ccccc3nc2NS(=O)(=O)c2cnn(C)c2)c(OCCC(C)O)c(OC)c1] |
| I-40 | 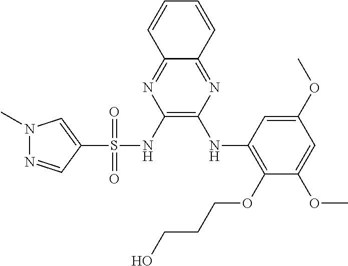 |
| I-41 | 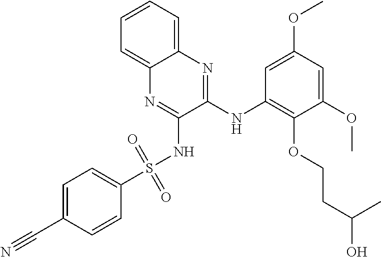 [SMILES: COc1cc(Nc2nc3ccccc3nc2NS(=O)(=O)c2ccc(C#N)cc2)c(OCCC(C)O)c(OC)c1] |
| I-42 | 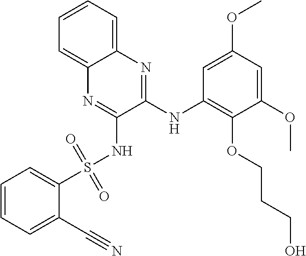 [SMILES: COc1cc(Nc2nc3ccccc3nc2NS(=O)(=O)c2ccccc2C#N)c(OCCCO)c(OC)c1] |
| I-43 | 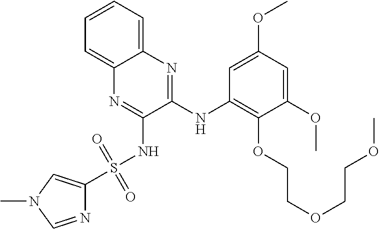 |
| I-44 | 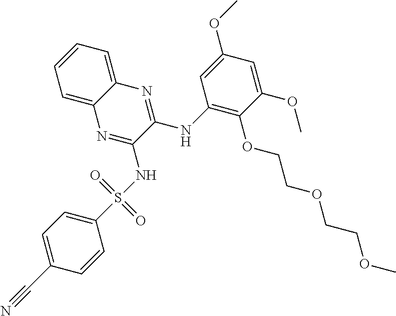 [SMILES: COCCOCCOc1c(Nc2nc3ccccc3nc2NS(=O)(=O)c2ccc(C#N)cc2)cc(OC)cc1OC] |
| I-45 | 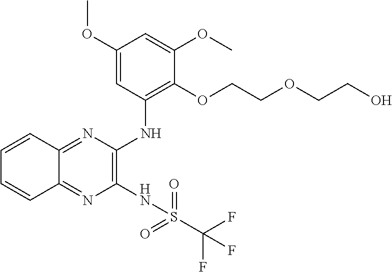 [SMILES: COc1cc(Nc2nc3ccccc3nc2NS(=O)(=O)C(F)(F)F)c(OCCOCCO)c(OC)c1] |
| I-46 | 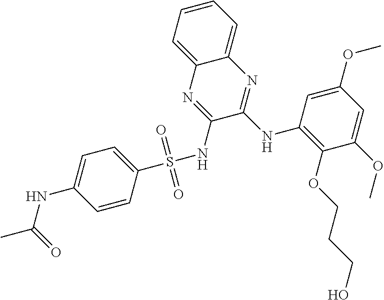 [SMILES: COc1cc(Nc2nc3ccccc3nc2NS(=O)(=O)c2ccc(NC(C)=O)cc2)c(OCCCO)c(OC)c1] |
| I-47 | 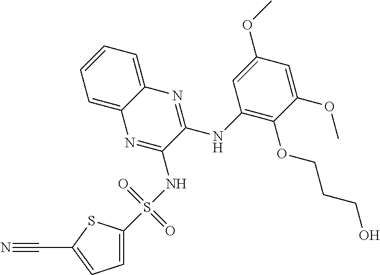 [SMILES: COc1cc(Nc2nc3ccccc3nc2NS(=O)(=O)c2ccc(C#N)s2)c(OCCCO)c(OC)c1] |
| I-48 | 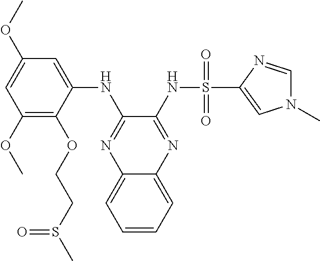 [SMILES: COc1cc(Nc2nc3ccccc3nc2NS(=O)(=O)c2cn(C)cn2)c(OCCS(C)=O)c(OC)c1] |
| I-49 | 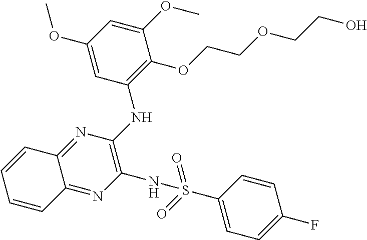 [SMILES: COc1cc(Nc2nc3ccccc3nc2NS(=O)(=O)c2ccc(F)cc2)c(OCCOCCO)c(OC)c1] |
| I-50 | 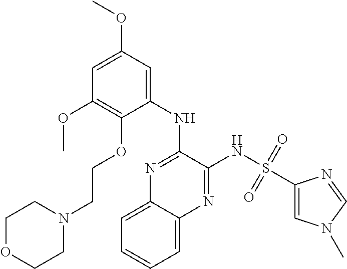 [SMILES: COc1cc(Nc2nc3ccccc3nc2NS(=O)(=O)c2cn(C)cn2)c(OCCN2CCOCC2)c(OC)c1] |
| I-51 | 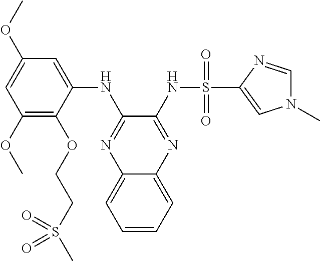 [SMILES: COc1cc(Nc2nc3ccccc3nc2NS(=O)(=O)c2cn(C)cn2)c(OCCS(C)(=O)=O)c(OC)c1] |
| I-52 | 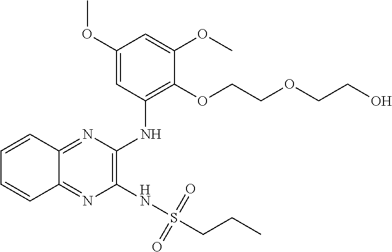 [SMILES: CCCS(=O)(=O)Nc1nc2ccccc2nc1Nc1cc(OC)cc(OC)c1OCCOCCO] |
| I-53 | 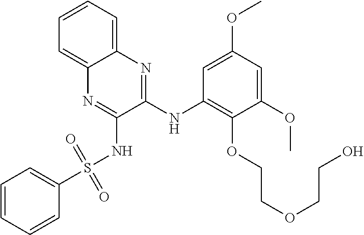 [SMILES: COc1cc(Nc2nc3ccccc3nc2NS(=O)(=O)c2ccccc2)c(OCCOCCO)c(OC)c1] |
| I-54 | 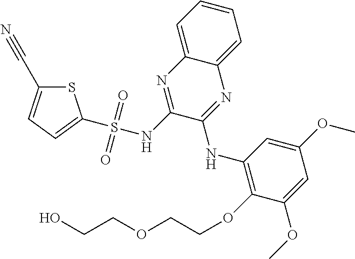 [SMILES: COc1cc(Nc2nc3ccccc3nc2NS(=O)(=O)c2ccc(C#N)s2)c(OCCOCCO)c(OC)c1] |
| I-55 | 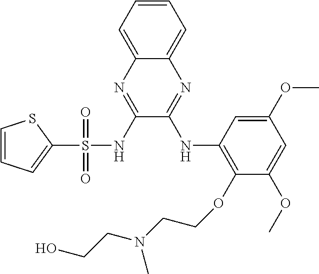 [SMILES: COc1cc(Nc2nc3ccccc3nc2NS(=O)(=O)c2cccs2)c(OCCN(C)CCO)c(OC)c1] |
| I-56 | 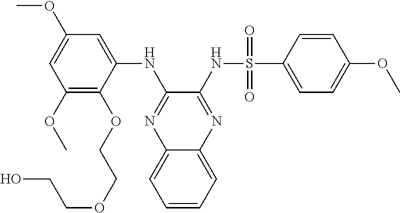 |
| I-57 | 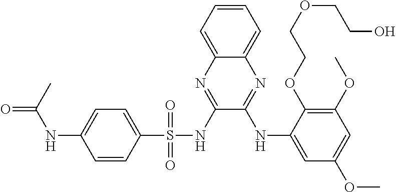 [SMILES: COc1cc(Nc2nc3ccccc3nc2NS(=O)(=O)c2ccc(NC(C)=O)cc2)c(OCCOCCO)c(OC)c1] |
| I-58 | 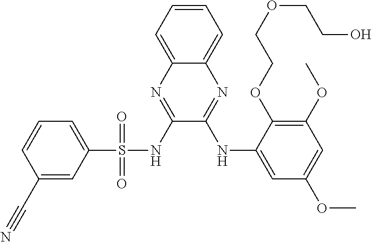 [SMILES: COc1cc(Nc2nc3ccccc3nc2NS(=O)(=O)c2cccc(C#N)c2)c(OCCOCCO)c(OC)c1] |
| I-59 | 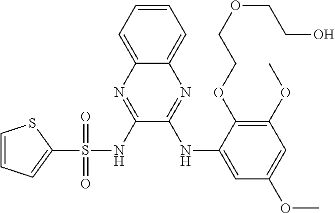 [SMILES: COc1cc(Nc2nc3ccccc3nc2NS(=O)(=O)c2cccs2)c(OCCOCCO)c(OC)c1] |
| I-60 | 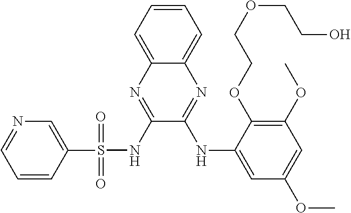 [SMILES: COc1cc(Nc2nc3ccccc3nc2NS(=O)(=O)c2cccnc2)c(OCCOCCO)c(OC)c1] |
| I-61 | 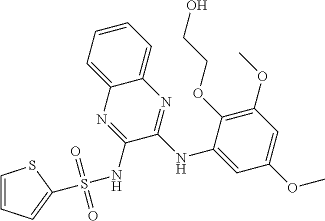 [SMILES: COc1cc(Nc2nc3ccccc3nc2NS(=O)(=O)c2cccs2)c(OCCO)c(OC)c1] |
| I-62 | 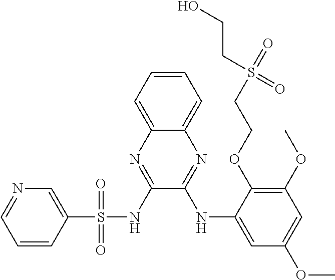 [SMILES: COc1cc(Nc2nc3ccccc3nc2NS(=O)(=O)c2cccnc2)c(OCCS(=O)(=O)CCO)c(OC)c1] |
| I-63 | 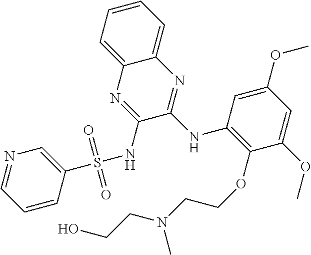 [SMILES: COc1cc(Nc2nc3ccccc3nc2NS(=O)(=O)c2cccnc2)c(OCCN(C)CCO)c(OC)c1] |
| I-64 | 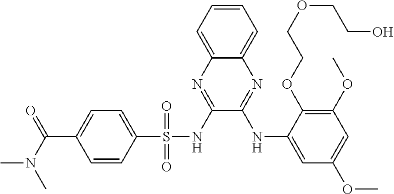 [SMILES: COc1cc(Nc2nc3ccccc3nc2NS(=O)(=O)c2ccc(C(=O)N(C)C)cc2)c(OCCOCCO)c(OC)c1] |
| I-65 | 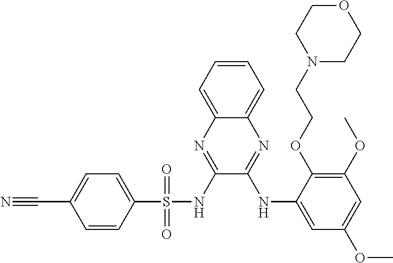 [SMILES: COc1cc(Nc2nc3ccccc3nc2NS(=O)(=O)c2ccc(C#N)cc2)c(OCCN2CCOCC2)c(OC)c1] |
| I-66 | 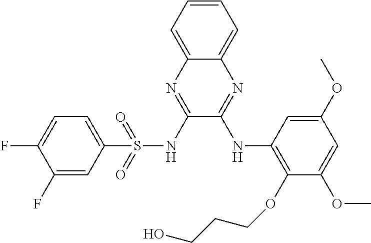 [SMILES: COc1cc(Nc2nc3ccccc3nc2NS(=O)(=O)c2ccc(F)c(F)c2)c(OCCCO)c(OC)c1] |
| I-67 | 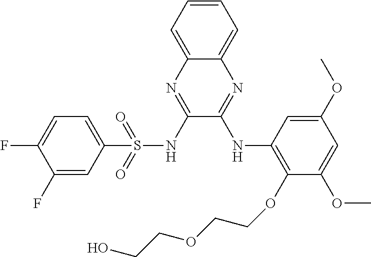 [SMILES: COc1cc(Nc2nc3ccccc3nc2NS(=O)(=O)c2ccc(F)c(F)c2)c(OCCOCCO)c(OC)c1] |
| I-68 | 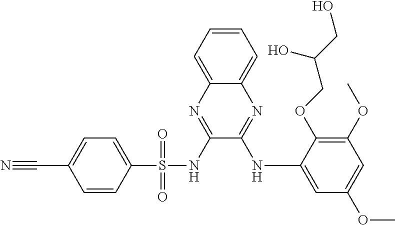 [SMILES: COc1cc(Nc2nc3ccccc3nc2NS(=O)(=O)c2ccc(C#N)cc2)c(OCC(O)CO)c(OC)c1] |
| I-69 | 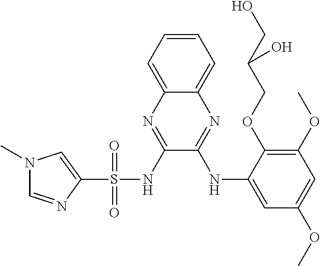 [SMILES: COc1cc(Nc2nc3ccccc3nc2NS(=O)(=O)c2cn(C)cn2)c(OCC(O)CO)c(OC)c1] |
| I-70 | 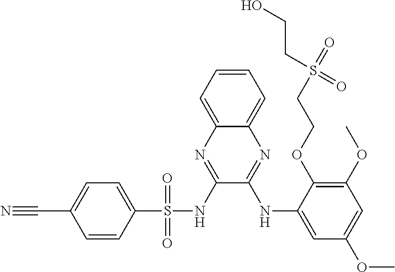 [SMILES: COc1cc(Nc2nc3ccccc3nc2NS(=O)(=O)c2ccc(C#N)cc2)c(OCCS(=O)(=O)CCO)c(OC)c1] |
| I-71 | 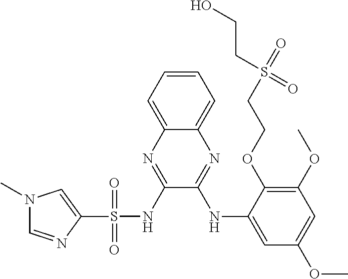 |
| I-72 | 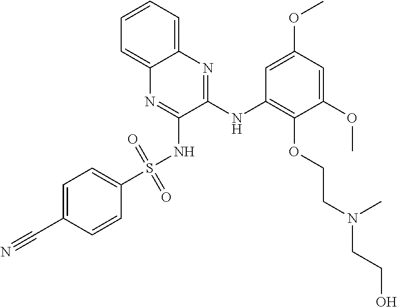 [SMILES: COc1cc(Nc2nc3ccccc3nc2NS(=O)(=O)c2ccc(C#N)cc2)c(OCCN(C)CCO)c(OC)c1] |
| I-73 | 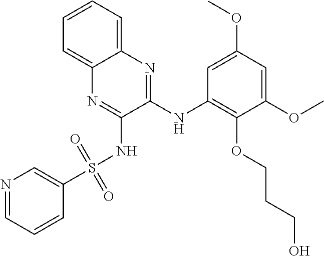 [SMILES: COc1cc(Nc2nc3ccccc3nc2NS(=O)(=O)c2cccnc2)c(OCCCO)c(OC)c1] |
| I-74 | 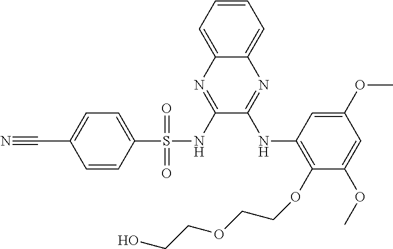 [SMILES: COc1cc(Nc2nc3ccccc3nc2NS(=O)(=O)c2ccc(C#N)cc2)c(OCCOCCO)c(OC)c1] |
| I-75 | 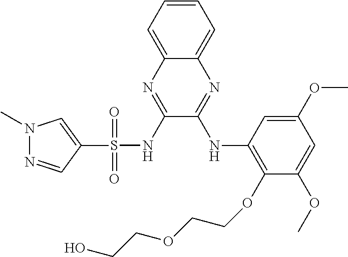 |
| I-76 | 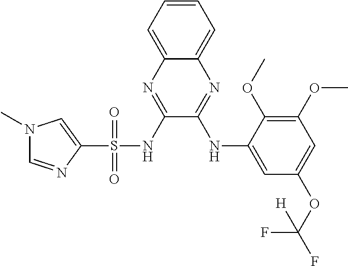 |
| I-77 | 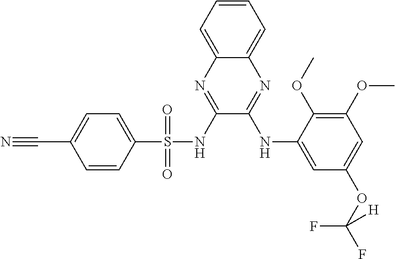 [SMILES: COc1cc(OC(F)F)cc(Nc2nc3ccccc3nc2NS(=O)(=O)c2ccc(C#N)cc2)c1OC] |
| I-78 | 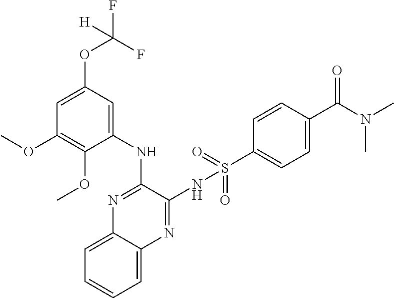 |
| I-79 | 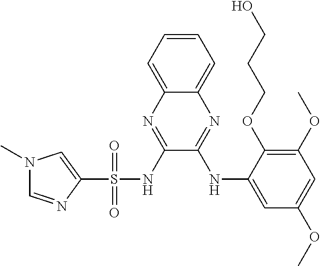 [SMILES: COc1cc(Nc2nc3ccccc3nc2NS(=O)(=O)c2cn(C)cn2)c(OCCCO)c(OC)c1] |
| I-80 | 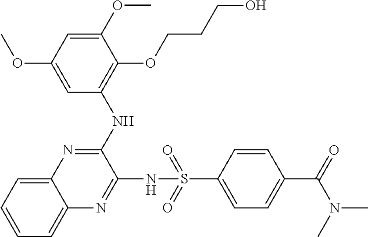 [SMILES: COc1cc(Nc2nc3ccccc3nc2NS(=O)(=O)c2ccc(C(=O)N(C)C)cc2)c(OCCCO)c(OC)c1] |
| I-81 | 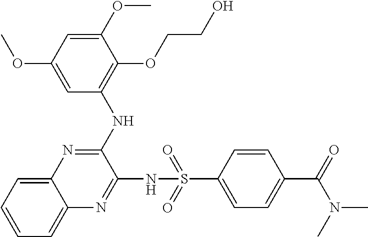 [SMILES: COc1cc(Nc2nc3ccccc3nc2NS(=O)(=O)c2ccc(C(=O)N(C)C)cc2)c(OCCO)c(OC)c1] |
| I-82 | 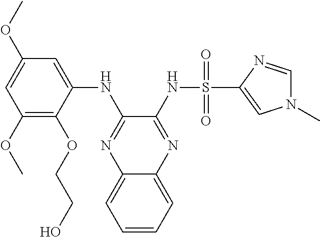 [SMILES: COc1cc(Nc2nc3ccccc3nc2NS(=O)(=O)c2cn(C)cn2)c(OCCO)c(OC)c1] |
| I-83 | 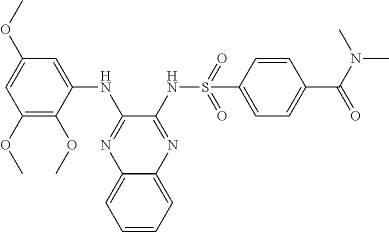 [SMILES: COc1cc(Nc2nc3ccccc3nc2NS(=O)(=O)c2ccc(C(=O)N(C)C)cc2)c(OC)c(OC)c1] |
| I-84 | 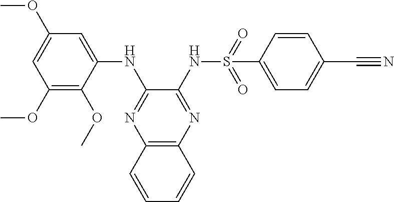 [SMILES: COc1cc(Nc2nc3ccccc3nc2NS(=O)(=O)c2ccc(C#N)cc2)c(OC)c(OC)c1] |
| I-85 | 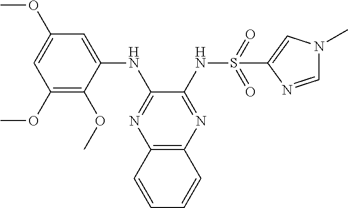 |
| I-86 | 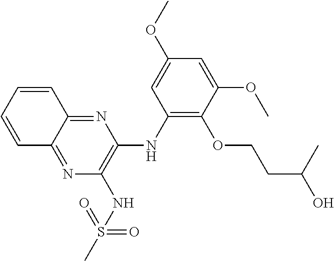 [SMILES: COc1cc(Nc2nc3ccccc3nc2NS(C)(=O)=O)c(OCCC(C)O)c(OC)c1] |
